(19) United States
(12) Patent Application Publication
(10) Pub. No.: US 2020/0079785 A1
(43) Pub. Date: Mar. 12, 2020

(54) MITOMYCIN C PRODRUG LIPOSOME FORMULATIONS AND USES THEREOF

(71) Applicant: MALLINCKRODT LLC, Hazelwood, MO (US)

(72) Inventors: William McGHEE, Fenton, MO (US); Michelle SCHMIDT, Millstadt, IL (US); Margaret GRAPPERHAUS, Troy, IL (US); Kah Tiong KUAN, Ballwin, MO (US)

(73) Assignee: Mallinckrodt LLC, Hazelwood, MO (US)

(21) Appl. No.: 16/348,433

(22) PCT Filed: Nov. 8, 2017
(86) PCT No.: PCT/US2017/060619

§ 371 Date: May 8, 2019

Related U.S. Application Data
(60) Provisional application No. 62/418,950, filed on Nov. 8, 2016.

(51) Int. Cl. C07D 487/14; A61K 9/127

(52) U.S. Cl. CPC C07D 487/14; A61K 9/1277; A61K 9/127

(57) ABSTRACT

The present invention provides MMC prodrug compounds and liposomal MMC prodrugs and compositions thereof for the treatment of cancer. The compositions include liposomes containing a phosphatidylcholine lipid, a sterol, a PEG-lipid and a MMC prodrug. The present invention also provides liposomal compositions for the treatment of cancer comprising administering to a patient in need thereof a liposome, wherein the liposome comprises: a phosphatidylcholine lipid; a sterol; a PEG-lipid and a MMC prodrug or a pharmaceutically-acceptable salt thereof.

### CLAIM OF PRIORITY

This application claims priority to U.S. Provisional Application No. 62/418,950 filed on Nov. 8, 2016. The entire content of the above-referenced application is incorporated by reference herein.

### BACKGROUND OF THE INVENTION

Mitomycin C (“MMC”) is a cytotoxic agent that has been shown to have antitumor activity. It has the following chemical formula:

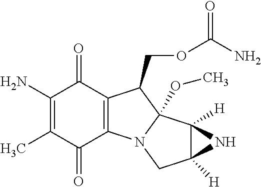 [SMILES: CO[C@@]12[C@H](COC(N)=O)C3=C(C(=O)C(C)=C(N)C3=O)N1C[C@@H]1N[C@@H]12]

MMC undergoes a reductive enzymatic activation to generate a bis-electophilic intermediate that preferentially akylates DNA and forms crosslinks between complimentary strands of DNA. In effect, it is a potent DNA crosslinker. This interaction prevents the separation of the complimentary DNA strands, thus inhibiting DNA replication and cell proliferation. MMC has been marketed under the brand name Mutamycin, and is FDA-approved for treatment of certain types of cancers, such as gastric and pancreatic adenocarcinomas in combination with certain other chemotherapeutic agents.

Despite its therapeutic advantages, MMC treatment is hindered by undesirable side effects, such as short half-life and toxicity. Because of its short half-life (alpha=8 minutes and terminal=48 minutes) and toxicity profile, it has not been approved as a monotherapy. Instead, it is utilized sparingly in combination with other approved chemotherapeutic agents for specific cancers and as a palliative treatment when other modalities have failed. Drug delivery systems for MMC date to the early 1980s. Strategies have encompassed dextran and other polymer conjugates with some success in achieving sustained release and reduced toxicity. Attempts to encapsulate MMC in liposomes passively or via cyclodextrin complex have resulted in low loading efficiency with concomitant rapid release.

Recently, Gabizon et al. have prepared lipid conjugates for subsequent insertion into the lipid bilayer of liposomes. Preclinical studies have demonstrated delivery and release of MCC in vivo, and this formulation has advanced to clinical trials as Promitil®, providing some validation for a liposomal approach. However, Promitil is a MMC lipid-based prodrug (MLP) (2,3-(distearoyloxy)propane-1-dithio-4′-benzyloxycarbonyl-MMC) whose structure is shown below:

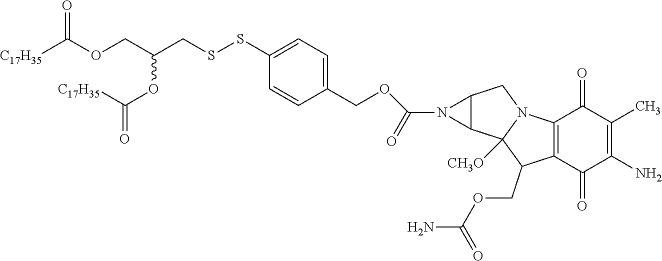

MLP is inserted into the liposome lipid bilayer via a lipid anchor. The external presentation of the dithiobenzyl may result in decreased serum stability and premature drug release due to potential cleavage of the disulfide linkage by biological reductants in circulation. Further, the loading of the liposome is dependent on, and limited by, the amount of drug that can be accommodated on the surface of the bilayer of the liposome. Thus, the size of MLP may limit the amount that can be loaded onto the liposome and may impact the stability of the liposome.

Liposomal encapsulation is still an attractive approach to significantly improve the exposure of MMC and to circumvent the dose-limiting toxicity (DLT) of acute and cumulative myelosuppression resulting in an overall improvement in the therapeutic index. Furthermore, MMC has been known to cause a form of nephrotoxicity known as hemolytic-uremic syndrome, and liposome encapsulation may alter the clearance mechanism and mitigate this toxicity. Towards this end, MMC prodrugs have been designed to incorporate functionality to facilitate remote loading into a liposome with surprising loading efficiency, extended release and improved efficacy, and reduced toxicity.

### BRIEF SUMMARY OF THE INVENTION

In one embodiment, the present invention provides MMC prodrugs. In a further embodiment, the MMC prodrug has the formula (I):

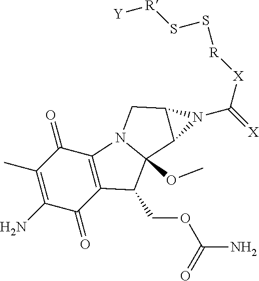

wherein R and R′ is independently selected from the group consisting of: an alkyl, such as (CH₂)ₙ, where n=1-6, —C₆H₄—, CH₂—C₆H₄—, and CH₂—C₆H₄—CH2—, and alkylaryl;
X is selected from the group consisting of: S, Se, and O; and
Y is selected from the group consisting of: a piperadinyl, a piperazinyl, a pyridinyl, dimethylamino, diethylamino, dipropylamino, morpholino, HO—CH₂CH₂NH—, (HO—CH₂CH₂)₂N—, HO—CH₂CH₂—N(Me)—, C₆H₄CH₂NH—, and C₆H₄CH₂N(Me).

In one embodiment, the present invention provides MMC prodrugs. In a further embodiment, the MMC prodrug has the formula:

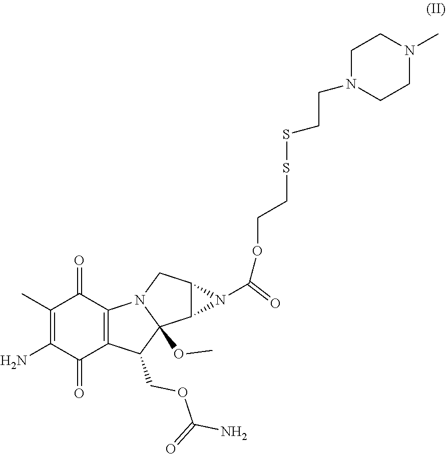

or a pharmaceutically-acceptable salt thereof.

In another embodiment, the MMC prodrug has the formula:

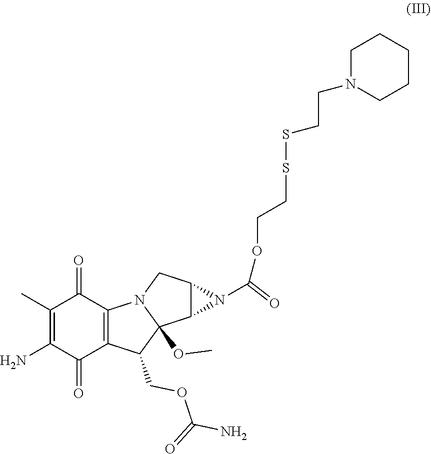 [SMILES: CO[C@@]12[C@H](COC(N)=O)C3=C(C(=O)C(C)=C(N)C3=O)N1C[C@H]1[C@@H]2N1C(=O)OCCSSCCN1CCCCC1]

or a pharmaceutically-acceptable salt thereof.

In a further embodiment, the MMC prodrug has the formula:

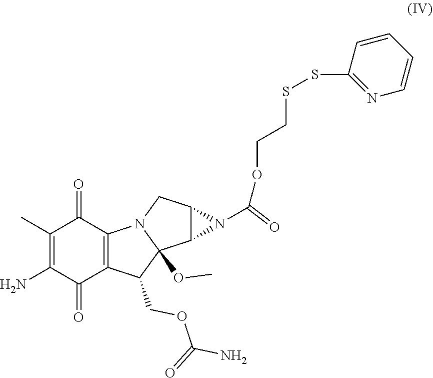 [SMILES: CO[C@@]12[C@H](COC(N)=O)C3=C(C(=O)C(C)=C(N)C3=O)N1C[C@H]1[C@@H]2N1C(=O)OCCSSc1ccccn1]

or a pharmaceutically-acceptable salt thereof.

In still another embodiment, MMC prodrugs are suitable for remote loading into the aqueous interior of the liposome. In some embodiments, the aqueous interior of the liposomal nanoparticles has an acidic pH relative to the external medium. In further embodiments, the MMC prodrugs reside within or is stably associated with the liposome membrane. In still a further embodiment, the MMC prodrugs are entrapped in the interior of the liposome via a thiol-containing compound, such as glutathione.

In yet another embodiment, the invention provides a method for preparing a liposomal MMC prodrug. The method includes: a) forming a first liposome having a lipid bilayer including a phosphatidylcholine lipid, a poly(ethylene glycol)-phospholipid conjugate (“PEG-lipid”) and a sterol, wherein the lipid bilayer encapsulates an interior compartment comprising an aqueous solution; and b) loading the first liposome with a MMC prodrug or a pharmaceutically-acceptable salt thereof, to form a loaded liposome. In a further embodiment, the interior compartment can further comprise a thiol-containing compound, such as glutathione.

In still another embodiment, the invention provides a method for preparing liposomal MMC prodrug. The method includes: a) forming a first liposome having a lipid bilayer including a phosphatidylcholine lipid and a sterol, wherein the lipid bilayer encapsulates an interior compartment comprising an aqueous solution, and, optionally, a thiol-containing compound, such as glutathione; b) loading the first liposome with a MMC prodrug, or a pharmaceutically-acceptable salt thereof, to form a loaded liposome; and c) forming a mixture containing the loaded liposome and a PEG-lipid under conditions sufficient to allow insertion of the PEG-lipid into the lipid bilayer.

In one embodiment, a pharmaceutical composition is provided comprising a liposomal MMC prodrug provided herein and a pharmaceutically acceptable excipient.

In a further embodiment, the present invention provides a pharmaceutical composition for the treatment of cancer. The pharmaceutical composition includes a liposome containing a phosphatidylcholine lipid, a sterol, a PEG-lipid, and a MMC prodrug or pharmaceutically- acceptable salts thereof In a further embodiment, the invention provides a method for treating cancer. The method includes administering to a patient in need thereof the pharmaceutical composition of the present invention.

### BRIEF DESCRIPTION OF THE DRAWINGS

FIG. 1 shows a synthetic scheme for the MMC prodrug of formula II.

FIG. 2 shows a synthetic scheme for the MMC prodrug of formula III.

FIG. 3 shows a synthetic scheme for the MMC prodrug of formula IV.

FIG. 4 shows a schematic for trapping the MMC prodrug of formula II in the interior of the liposome via glutathione.

FIG. 5 shows the release of glutathione-entrapped liposomal MMC prodrug MP-3861 and its conversion to MMC in fetal bovine serum.

FIG. 6 shows the release profile of liposomal MMC prodrug MP-3854 in fetal bovine serum as a function of the buffer and pH.

FIG. 7 shows the anti-tumor activity of liposomal MMC prodrugs and free MMC against HT29 colorectal adenocarcinoma xenografts in athymic nude mice. Mean tumor volume (mm³) over time is shown after a single IV administration of MMC, liposomal MMC prodrugs, or saline. Data are represented as mean±standard error (n=10).

FIG. 8A shows the change in body weight in athymic nude mice bearing HT29 colorectal adenocarcinoma xenografts after a single IV administration of MMC, liposomal MMC prodrugs, or saline. FIG. 8B shows the Kaplan-Meier survival plot over time in athymic nude mice bearing HT29 colorectal adenocarcinoma xenografts after a single IV administration of MMC, liposomal MMC prodrugs, or saline. Data are represented as mean±standard error (n=10).

FIG. 9A shows the mean tumor volume (mm³) in athymic nude mice bearing A549 lung carcinoma xenografts after a single IV administration of MMC, liposomal MMC prodrug, or saline. FIG. 9B shows the mean tumor volume (mm³) in athymic nude mice bearing A549 lung carcinoma xenografts after a single IV administration of MMC, liposomal MMC prodrug, or saline. FIG. 9C shows the percent change in body weight in athymic nude mice bearing A549 lung carcinoma xenografts after a single IV administration of MMC, liposomal MMC prodrug, or saline. FIG. 9D shows the Kaplan-Meier survival plot over time in athymic nude mice bearing A549 lung carcinoma xenografts after a single IV administration of MMC, liposomal MMC prodrug, or saline. Data are represented as mean±standard error (n=10).

FIG. 10A shows the mean tumor volume (mm³) in athymic nude mice bearing HT29 colorectal adenocarcinoma xenografts after a single IV administration of liposomal MMC prodrug or saline. FIG. 10B shows the percent change in body weight over time in athymic nude mice bearing HT29 colorectal adenocarcinoma xenografts after a single IV administration of liposomal MMC prodrug or saline. Data are represented as mean±standard error (n=5).

FIG. 11A shows the mean tumor volume (mm³) in athymic nude mice bearing HT29 colorectal adenocarcinoma xenografts after a single IV administration of MMC, liposomal MMC prodrug, or saline. FIG. 11B shows the percent change in body weight over time in athymic nude mice bearing HT29 colorectal adenocarcinoma xenografts after a single IV administration of liposomal MMC prodrug, or saline. Data are represented as mean±standard error (n=5).

### DETAILED DESCRIPTION OF THE INVENTION

### I. General

The present invention provides novel MMC prodrugs and liposomal formulations and pharmaceutical compositions thereof. The MMC prodrug liposomal formulations described herein demonstrate several advantages including increases in in vivo circulation time (i.e., half-life) and efficacy, and reduced toxicity. The MMC prodrugs and liposomal formulations are useful for the treatment of cancer as described herein.

### II. Definitions

As used herein, the term “liposome” encompasses any compartment enclosed by a lipid bilayer. The term liposome includes unilamellar vesicles which are comprised of a single lipid bilayer and generally have a diameter in the range of about 20 to about 400 nm. Liposomes can also be multilamellar, which generally have a diameter in the range of 1 to 10 μm. In some embodiments, liposomes can include multilamellar vesicles (MLVs; from about 1 μm to about 10 μm in size), large unilamellar vesicles (LUVs; from a few hundred nanometers to about 10 μm in size), and small unilamellar vesicles (SUVs; from about 20 nm to about 200 nm in size).

As used herein, the term “phosphatidylcholine lipid” refers to a diacylglyceride phospholipid having a choline headgroup (i.e., a 1,2-diacyl-sn-glycero-3-phosphocholine). The acyl groups in a phosphatidylcholine lipid are generally derived from fatty acids having from 6 to 24 carbon atoms. Phosphatidylcholine lipids can include synthetic and naturally-derived 1,2-diacyl-sn-glycero-3 -phosphocholines.

As used herein, the term “sterol” refers to a steroid containing at least one hydroxyl group. A steroid is characterized by the presence of a fused, tetracyclic gonane ring system. Sterols include, but are not limited to, cholesterol (i.e., 2,15-dimethyl-14-(1,5-dimethylhexyl) tetracyclo[8.7.0.0_{2,7}.0^{11,15}]heptacos-7-en-5-ol; Chemical Abstracts Services Registry No. 57-88-5).

As used herein, the term “PEG-lipid” refers to a poly(ethylene glycol) polymer covalently bound to a hydrophobic or amphipilic lipid moiety. The lipid moiety can include fats, waxes, steroids, fat-soluble vitamins, monoglycerides, diglycerides, phospholipids, and sphingolipids. Preferred PEG-lipids include diacyl-phosphatidylethanolamine-N-[methoxy(polyethene glycol)]s and N-acyl-sphingosine-1-{succinyl[methoxy(polyethylene glycol)]}s. The molecular weight of the PEG in the PEG-lipid is generally from about 500 to about 5000 Daltons (Da; g/mol). The PEG in the PEG-lipid can have a linear or branched structure.

As used herein, the terms “molar percentage” and “mol %” refer to the number of moles of a given lipid component of a liposome divided by the total number of moles of all lipid components. Unless explicitly stated, the amounts of active agents, diluents, or other components are not included when calculating the mol % for a lipid component of a liposome.

As used herein, the term “loading” refers to effecting the accumulation of a MMC prodrug in a liposome. The MMC prodrug can be encapsulated in the aqueous interior of the liposome, or it can be embedded in the lipid bilayer. Liposomes can be passively loaded, wherein the MMC prodrug is included in the solutions used during liposome preparation. Alternatively, liposomes can be remotely loaded by establishing a chemical gradient (e.g, a pH or ion gradient) across the liposome bilayer, causing migration of the MMC prodrug from the aqueous exterior to the liposome interior compartment.

As used herein, the term “composition” refers to a product comprising the specified ingredients in the specified amounts, as well as any product which results, directly or indirectly, from the combination of the specified ingredients in the specified amounts. Pharmaceutical compositions of the present invention generally contain a liposomal MMC prodrug as described herein and a pharmaceutically acceptable carrier, diluent, or excipient. By “pharmaceutically acceptable,” it is meant that the carrier, diluent, or excipient must be compatible with the other ingredients of the formulation and non-deleterious to the recipient thereof.

As used herein, the term “cancer” refers to conditions including human cancers and carcinomas, sarcomas, adenocarcinomas, lymphomas, leukemias, and solid and lymphoid cancers. Examples of different types of cancer include, but are not limited to, lung cancer (e.g., non-small cell lung cancer or NSCLC), ovarian cancer, prostate cancer, colorectal cancer, liver cancer (i.e., hepatocarcinoma), renal cancer (i.e., renal cell carcinoma), bladder cancer, breast cancer, thyroid cancer, pleural cancer, pancreatic cancer, uterine cancer, cervical cancer, testicular cancer, anal cancer, pancreatic cancer, bile duct cancer, gastrointestinal carcinoid tumors, esophageal cancer, gall bladder cancer, appendix cancer, small intestine cancer, stomach (gastric) cancer, cancer of the central nervous system, cancer of unknown primary origin, skin cancer, choriocarcinoma, head and neck cancer, blood cancer, osteogenic sarcoma, fibrosarcoma, neuroblastoma, glioma, melanoma, B-cell lymphoma, non-Hodgkin's lymphoma, Burkitt's lymphoma, small cell lymphoma, large cell lymphoma, monocytic leukemia, myelogenous leukemia, acute lymphocytic leukemia, acute myelocytic leukemia, and multiple myeloma.

As used herein, the terms “treat,” “treating,” and “treatment” refer to any indicia of success in the treatment or amelioration of a cancer or a symptom of cancer, including any objective or subjective parameter such as abatement; remission (e.g., full or partial); achieving a complete response in a patient; achieving a partial response in a patient; maintaining a stable disease state (e.g., the target lesions have not decreased in size, however, the target lesions have also not increased in size and/or new lesions have not formed); diminishing of symptoms or making the cancer or cancer symptom more tolerable to the patient (clinical benefit). The treatment or amelioration of symptoms can be based on any objective or subjective parameter, including, e.g., the result of a physical examination (clinical benefit) or clinical test.

As used herein, the terms “administer,” “administered,” or “administering” refer to methods of administering the liposome compositions of the present invention. The liposome compositions of the present invention can be administered in a variety of ways, including parenterally, intravenously, intradermally, intramuscularly, or intraperitoneally. The liposome compositions can also be administered as part of a composition or formulation.

As used herein, the term “subject” refers to any mammal, in particular a human, at any stage of life.

The term “half-life” or “t_{1/2}” as used herein refers to the amount of time required for the concentration or amount of the drug found in the blood or plasma to decrease by one-half. This decrease in drug concentration is a reflection of its metabolism plus excretion or elimination after absorption is complete and distribution has reached an equilibrium or quasi equilibrium state. The half-life of a drug in the blood may be determined graphically off of a pharmacokinetic plot of a drug's blood-concentration time plot, typically after intravenous administration to a sample population. The half-life can also be determined using mathematical calculations that are well known in the art. Further, as used herein, the term “half-life” also includes the “apparent half-life” of a drug. The apparent half-life may be a composite number that accounts for contributions from other processes besides elimination, such as absorption, reuptake, or enterohepatic recycling.

The term “AUC” means an area under the drug concentration-time curve.

The term “AUCₐₗₗ” means an area under the drug concentration-time curve up to the last time-point below the limit of quantitation.

The term “Partial AUC” means an area under the drug concentration-time curve (AUC) calculated using linear trapezoidal summation for a specified interval of time, for example, AUC₍₀₋₁ₕᵣ₎, AUC₍₀₋₂ₕᵣ₎, AUC₍₀₋₃ₕᵣ₎, AUC₍₀₋₄ₕᵣ₎, AUC₍₀₋₅ₕᵣ₎, AUC₍₀₋₆ₕᵣ₎, AUC₍₀₋₇ₕᵣ₎, AUC₍₀₋₈ₕᵣ₎, AUC₍₀₋₉ₕᵣ₎, AUC₍₀₋₁₀ₕᵣ₎, AUC₍₀₋₁₁ₕᵣ₎, AUC₍₀₋₁₂ₕᵣ₎, AUC_{(0-(Tmax of IR product+2SD))}, AUC₍₀₋₍ₓ₎ₕᵣ₎, AUC_{(x-yhr)}, AUC_{(Tmax-t)}, AUC₍₀₋₍ₜ₎ₕᵣ₎, AUC_{(Tmax of IR product+2SD)-t)}, or AUC_{(0-∞)}.

The term “Cₘₐₓ” refers to the maximum plasma concentration obtained during a dosing interval.

The use of individual numerical values are stated as approximations as though the values were preceded by the word “about” or “approximately.” Similarly, the numerical values in the various ranges specified in this application, unless expressly indicated otherwise, are stated as approximations as though the minimum and maximum values within the stated ranges were both preceded by the word “about” or “approximately.” In this manner, variations above and below the stated ranges can be used to achieve substantially the same results as values within the ranges. As used herein, the terms “about” and “approximately” when referring to a numerical value shall have their plain and ordinary meanings to a person of ordinary skill in the art to which the disclosed subject matter is most closely related or the art relevant to the range or element at issue. The amount of broadening from the strict numerical boundary depends upon many factors. For example, some of the factors which may be considered include the criticality of the element and/or the effect a given amount of variation will have on the performance of the claimed subject matter, as well as other considerations known to those of skill in the art. As used herein, the use of differing amounts of significant digits for different numerical values is not meant to limit how the use of the words “about” or “approximately” will serve to broaden a particular numerical value or range. Thus, as a general matter, “about” or “approximately” broaden the numerical value. Also, the disclosure of ranges is intended as a continuous range including every value between the minimum and maximum values plus the broadening of the range afforded by the use of the term “about” or “approximately.” Consequently, recitation of ranges of values herein are merely intended to serve as a shorthand method of referring individually to each separate value falling within the range, unless otherwise indicated herein, and each separate value is incorporated into the specification as if it were individually recited herein.

### III. Embodiments of the Invention

In one embodiment, the present invention provides MMC prodrugs or pharmaceutical-acceptable salts thereof. In another embodiment, the MMC prodrugs are suitable for remote loading into the aqueous interior of a liposome. In another embodiment, the MMC prodrugs are entrapped in the interior of the liposome via a thiol-containing compound, for example, glutathione.

In another embodiment, the present invention provides a composition for the treatment of cancer. The composition includes a liposome containing a phosphatidylcholine lipid, a sterol, a PEG-lipid, and a MMC prodrug or a pharmaceutically-acceptable salt thereof; and a pharmaceutically-acceptable excipient.

In another embodiment, the invention provides liposomal compositions for the treatment of cancer comprising administering to a patient in need thereof a liposomal formulation, wherein the liposome comprises: a phosphatidylcholine lipid; a sterol; a PEG-lipid; and a MMC prodrug or a pharmaceutically-acceptable salt thereof.

In still another embodiment, the invention provides a method for preparing MMC prodrug liposomal formulations. The method includes: a) forming a first liposome having a lipid bilayer including a phosphatidylcholine lipid, PEG-lipid and a sterol, wherein the lipid bilayer encapsulates an interior compartment comprising an aqueous solution and, optionally a thiol-containing compound, such as glutathione; and b) loading the first liposome with a MMC prodrug, or a pharmaceutically-acceptable salt thereof, to form a loaded liposome. In a further embodiments, the PEG-lipid may be inserted into the lipid bilayer post-loading of the liposome with the MMC prodrug.

### A. MMC Prodrugs

In some embodiments, the MMC prodrug is a compound according to Formula I, or a pharmaceutically-acceptable salt thereof:

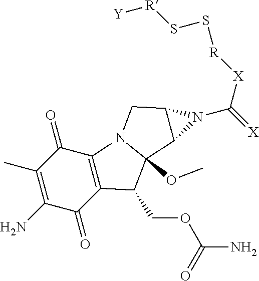

wherein R and R′ is independently selected from the group consisting of: an alkyl, such as (CH₂)ₙ, where n=1-6, —C₆H₄—, CH₂—C₆H₄—, and CH₂—C₆H₄—CH₂—, and alkylaryl;
X is selected from the group consisting of: S, Se, and O; and
Y is selected from the group consisting of: a piperadinyl, a piperazinyl, a pyridinyl, dimethylamino, diethylamino, dipropylamino, morpholino, HO—CH₂CH₂NH—, (HO—CH₂CH₂)₂N—, HO—CH₂CH₂—N(Me)—, C₆H₄CH₂NH—, and C₆H₄CH₂N(Me).

In some embodiments, the MMC prodrug is a compound according to Formula II, or a pharmaceutically-acceptable salt thereof:

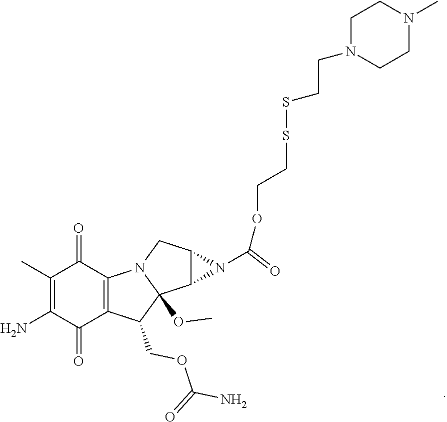

In some embodiments, the MMC prodrug is a compound according to Formula III, or a pharmaceutically-acceptable salt thereof:

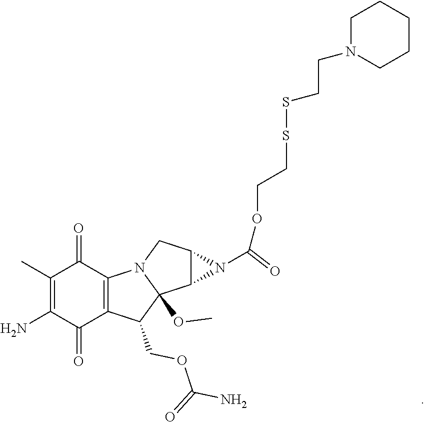 [SMILES: CO[C@@]12[C@H](COC(N)=O)C3=C(C(=O)C(C)=C(N)C3=O)N1C[C@H]1[C@@H]2N1C(=O)OCCSSCCN1CCCCC1]

In some embodiments, the MMC prodrug has the following Formula IV, or a pharmaceutically-acceptable salt thereof:

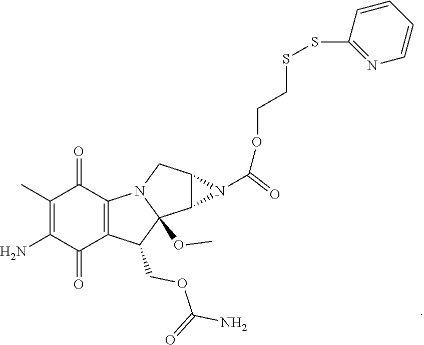 [SMILES: CO[C@@]12[C@H](COC(N)=O)C3=C(C(=O)C(C)=C(N)C3=O)N1C[C@H]1[C@@H]2N1C(=O)OCCSSc1ccccn1]

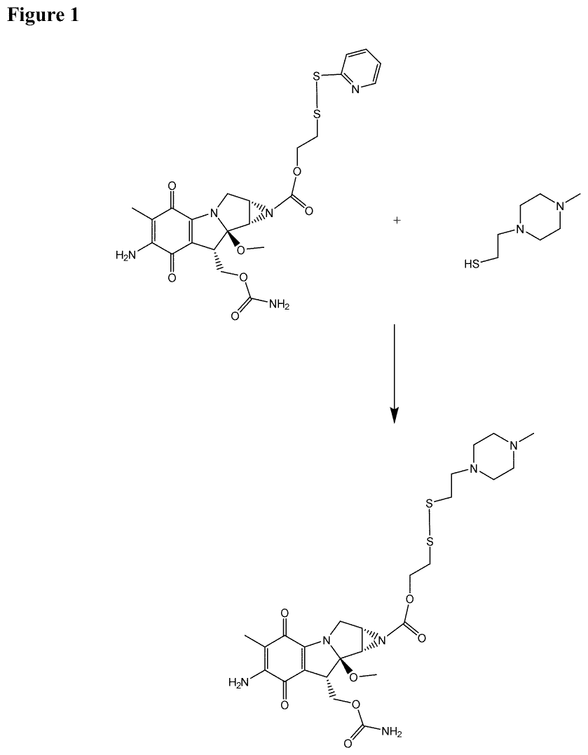

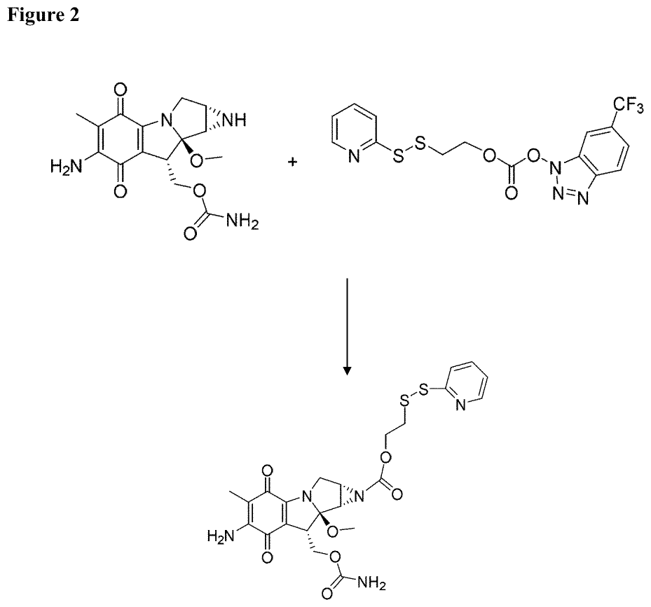

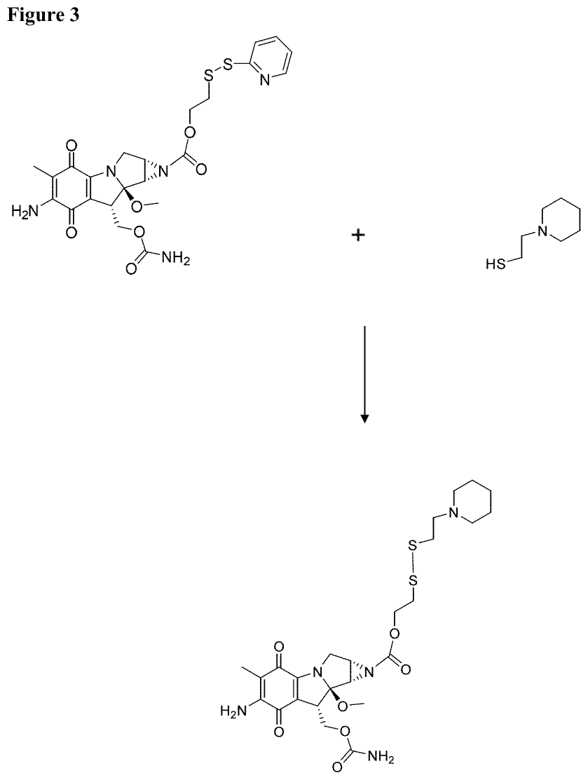

MMC prodrugs of Formula II, III and IV are synthesized as shown in FIGS. 1 to 3, respectively. MMC prodrugs are useful as chemotherapeutic agents for the treatment of various cancers, including, for example, stomach cancer, pancreatic cancer, breast cancer, ovarian cancer, lung cancer, bladder cancer, cervical cancer, colorectal cancer, anal cancer, esophogeal cancer, prostate cancer, liver cancer, and head and neck cancer.

As described above, the use of MMC for therapeutic purposes has been hampered by undesirable side effects, such as limited half-life and high toxicity. Various strategies have been employed to remedy these drawbacks. For example, Gabizon et al. have prepared lipid conjugates of MMC for subsequent insertion into the lipid bilayer of liposomes (see, e.g., U.S. Pat. No. 6,365,179). However, as discussed supra, the liposome of Gabizon may have limited drug loading and stability.

Incorporation of MMC into liposomes can improve half-life and reduce the toxicity of the MMC. However, the acid lability of MMC poses problems for purification and liposomal loading. In the present invention, a weak base or weak acid is conjugated to MMC via a cleavable disulfide linkage (i.e., MMC prodrug), which facilitates the remote loading into the aqueous interior of a liposome. This conjugation also provides for a more stable MMC structure by conversion of the nitrogen of the aziridine ring to a carbamate. In general, the weak base moiety can include an ionizable amino group, such as a pyridine group or a piperidino group, or a diamino group, such as a piperazino group.

The MMC prodrug will undergo triggered self-immolative elimination reaction to regenerate MMC at a target site, such as for example, a tumor site. The MMC prodrug can be converted at the disulfide bond to the corresponding thiol by, for example, an endogenous thiol containing compound via a disulfide exchange. Once conversion of the disulfide to the thiol occurs the intra-molecular elimination takes place as shown below.

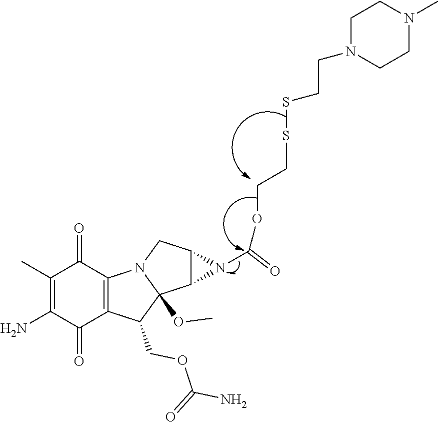

Conversion of the disulfide to a thiolate can occur via a reduction type mechanism, which will also facilitate elimination, to regenerate MMC.

### B. Liposomes

The liposomes of the present invention can contain any suitable lipid, including cationic lipids, zwitterionic lipids, neutral lipids, or anionic lipids as described above. Suitable lipids can include fats, waxes, steroids, cholesterol, fat-soluble vitamins, monoglycerides, diglycerides, phospholipids, sphingolipids, glycolipids, cationic or anionic lipids, derivatized lipids, and the like.

In general, the liposomes of the present invention contain at least one phosphatidylcholine (PC) lipid. Suitable PC lipids include saturated PCs and unsaturated PCs.

Examples of saturated PCs include, but are not limited to, 1,2-dilauroyl-sn-glycero-3-phosphocholine (DLPC), 1,2-dimyristoyl-sn-glycero-3-phosphocholine (dimyristoylphosphatidylcholine; DMPC), 1,2-distearoyl-sn-glycero-3-phosphocholine (distearoylphosphatidylcholine; DSPC), 1,2-dioleoyl-sn-glycero-3-phosphocholine (DOPC), 1,2-dipalmitoyl-sn-glycero-3-phosphocholine (dipalmitoylphosphatidylcholine; DPPC), 1-myristoyl-2-palmitoyl-sn-glycero-3-phosphocholine (MPPC), 1-palmitoyl-2-myristoyl-sn-glycero-3-phosphocholine (PMPC), 1-myristoyl-2-stearoyl-sn-glycero-3-phosphocholine (MSPC), 1-palmitoyl-2-stearoyl-sn-glycero-3-phosphocholine (PSPC), 1-stearoyl-2-palmitoyl-sn-glycero-3-phosphocholine (SPPC), and 1-stearoyl-2-myristoyl-sn-glycero-3-phosphocholine (SMPC).

Examples of unsaturated PCs include, but are not limited to, 1,2-dimyristoleoyl-sn-glycero-3-phosphocholine, 1,2-dimyristelaidoyl-sn-glycero-3-phosphocholine, 1,2-dipamiltoleoyl-sn-glycero-3-phosphocholine, 1,2-dipalmitelaidoyl-sn-glycero-3-phosphocholine, 1,2-dioleoyl-sn-glycero-3-phosphocholine (DOPC), 1,2-dielaidoyl-sn-glycero-3-phosphocholine, 1,2-dipetroselenoyl-sn-glycero-3-phosphocholine, 1,2-dilinoleoyl-sn-glycero-3-phosphocholine, 1-palmitoyl-2-oleoyl-sn-glycero-3-phosphocholine (palmitoyloleoylphosphatidylcholine; POPC), 1-palmitoyl-2-linoleoyl-sn-glycero-3-phosphocholine, 1-stearoyl-2-oleoyl-sn-glycero-3-phosphocholine (SOPC), 1-stearoyl-2-linoleoyl-sn-glycero-3-phosphocholine, 1-oleoyl-2-myristoyl-sn-glycero-3-phosphocholine (OMPC), 1-oleoyl-2-palmitoyl-sn-glycero-3-phosphocholine (OPPC), and 1-oleoyl-2-stearoyl-sn-glycero-3-phosphocholine (DSPC).

Lipid extracts, such as egg PC, heart extract, brain extract, liver extract, soy PC, and hydrogenated soy PC (HSPC), are also useful in the present invention.

In some embodiments, the liposome compositions will consist essentially of a PC lipid or mixture of PC lipids, cholesterol, a PEG-lipid, and a MMC prodrug. In still other embodiments, the liposome compositions will consist essentially of a single type of PC lipid, cholesterol, a PEG-lipid, and a MMC prodrug. In some embodiments, when a single type of PC lipid is used, it is selected from DOPC, DSPC, HSPC, DPPC, POPC, or SOPC.

In some embodiments, the PC lipid is selected from the group consisting of DPPC, DSPC, HSPC, and mixtures thereof. In some embodiments, the PC lipid is DSPC. In some embodiments, the compositions of the present invention include liposomes containing 45-75 mol % of a PC lipid or mixture of PC lipids, 50-70 mol % of a PC lipid or mixture of PC lipids, 50-65 mol % of a PC lipid or mixture of PC lipids, 50-60 mol % of a PC lipid or mixture of PC lipids, 50-56 mol % of a PC lipid or mixture of PC lipids, or 53-56 mol % of a PC lipid or mixture of PC lipids. The liposomes can contain, for example, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, or 75 mol % of a PC lipid or mixture of PC lipids. In still other embodiments, the liposomes contain about 55 mol % of a PC lipid or mixture of PC lipids. In additional embodiments, the liposomes contain about 54 mol % of a PC lipid or mixture of PC lipids. In further embodiments, the liposomes contain about 53 mol % of a PC lipid or mixture of PC lipids.

Other suitable phospholipids, generally used in low amounts or in amounts less than the PC lipids, include phosphatidic acids (PAs), phosphatidylethanolamines (PEs), phosphatidylglycerols (PGs), phosphatidylserine (PSs), and phosphatidylinositol (PIs). Examples of phospholipids include, but are not limited to, dimyristoylphosphatidylglycerol (DMPG), distearoylphosphatidylglycerol (DSPG), dioleoylphosphatidylglycerol (DOPG), dipalmitoylphosphatidylglycerol (DPPG), dimyristoylphosphatidylserine (DMPS), distearoylphosphatidylserine (DSPS), dioleoylphosphatidylserine (DOPS), dipalmitoylphosphatidylserine (DPPS), dioleoylphosphatidylethanolamine (DOPE), palmitoyloleoylphosphatidylcholine (POPC), palmitoyloleoylphosphatidylethanolamine (POPE), dipalmitoylphosphatidylethanolamine (DPPE), dimyristoylphosphoethanolamine (DMPE), distearoylphosphatidylethanolamine (DSPE), 16-O-monomethyl PE, 16-O-dimethyl PE, 18-1-trans PE, 1-stearoyl-2-oleoyl-phosphatidyethanolamine (SOPE), dielaidoylphosphoethanolamine (transDOPE), and cardiolipin.

Liposomes of the present invention can contain steroids, characterized by the presence of a fused, tetracyclic gonane ring system. Examples of steroids include, but are not limited to, cholic acid, progesterone, cortisone, aldosterone, testosterone, dehydroepiandrosterone, and sterols such as estradiol and cholesterol. Synthetic steroids and derivatives thereof are also contemplated for use in the present invention.

In general, the liposomes contain at least one sterol. In some embodiments, the sterol is cholesterol (i.e., 2,15-dimethyl-14-(1,5-dimethylhexyl)tetracyclo[8.7.0.0^{2,7}.0^{11,15}]heptacos-7-en-5-ol). In some embodiments, the liposomes can contain about 20-50 mol % of cholesterol, about 30-45 mol % of cholesterol, about 30-40 mol % of cholesterol, about 40-45 mol % of cholesterol or about 40-50 mol % of cholesterol. The liposomes can contain, for example, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 mol % of cholesterol.

The liposomes of the present invention can include any suitable PEG-lipid. In some embodiments, the PEG-lipid is a diacyl-phosphatidylethanolamine-N-[methoxy(polyethene glycol)]. The molecular weight of the poly(ethylene glycol) in the PEG-lipid is generally in the range of from about 500 Da to about 5000 Da. The poly(ethylene glycol) can have a molecular weight of, for example, 500 Da, 750 Da, 1000 Da, 2000 Da, or 5000 Da. In some embodiments, the PEG-lipid is selected from distearoyl-phosphatidylethanolamine-N-[methoxy(polyethene glycol)-2000] (DSPE-PEG-2000) and distearoyl-phosphatidylethanolamine-N-[methoxy (polyethene glycol)-5000] (DSPE-PEG-5000). In some embodiments, the PEG-lipid is DSPE-PEG-2000.

In general, the compositions of the present invention include liposomes containing 1-10 mol % of a PEG-lipid. The liposomes can contain, for example, 1, 2, 3, 4, 5, 6, 7, 8, 9, or 10 mol % of a PEG-lipid. In some embodiments, the liposomes containing 2-8 mol % of a PEG-lipid. In some embodiments, the liposomes contain 3-6 mol % of a PEG-lipid. In some embodiments, the liposomes contain 5 mol % of a PEG-lipid. In some embodiments, the liposomes contain 5 mol % of DSPE-PEG2000.

The liposomes of the present invention can also include some amounts of cationic lipids, which are generally in amounts lower than the amount of PC lipid. Cationic lipids contain positively charged functional groups under physiological conditions. Cationic lipids include, but are not limited to, N,N-dioleyl-N,N-dimethylammonium chloride (DODAC), N,N-distearyl-N,N-dimethylammonium bromide (DDAB), N-(1 -(2,3 -dioleoyloxy)propyl)-N,N,N-trimethylammonium chloride (DOTAP), N-(1-(2,3 -dioleyloxy)propyl)-N,N,N-trimethylammonium chloride (DOTMA), N-[1-(2,3,-ditetradecyloxy)propyl]-N,N-dimethyl-N-hydroxyethylammonium bromide (DMRIE), N-[1-(2,3,dioleyloxy)propyl]-N,N-dimethyl-N-hydroxy ethylammonium bromide (DORIE), 3β-[N-(N′,N′-dimethylaminoethane) carbamoyl] cholesterol (DC-Chol), dimethyldioctadecylammonium (DDAB), and N,N-dimethyl-2,3 -dioleyloxy)propylamine (DODMA).

In some embodiments of the present invention, the liposome includes from about 50 mol % to about 75 mol % of DSPC and from about 20 mol % to about 45 mol % of cholesterol. In some embodiments, the liposome includes about 55 mol % of DSPC, about 45 mol % of cholesterol, and about 5 mol % of DSPE-PEG-2000. In some embodiments, the liposome includes about 53 mol % of DSPC, about 44 mol % of cholesterol, and about 3 mol % of DSPE-PEG-2000. In some embodiments, the liposome includes about 66 mol % of DSPC, about 30 mol % of cholesterol, and about 4 mol % of DSPE-PEG-2000.

### C. Liposomal Glutathione Trapping

Liposomal drug leakage affects the shelf-life of liposomes. Drug leakage depends on the liposome composition and the physiochemistry of the drug. Large polar or ionic, water-soluble drugs are generally retained much more effectively than low molecular weight, amphiphilic compounds. Charged drugs may interact with the oppositely charged bilayer, which increases the encapsulation efficiency compared to drugs that do not interact with the bilayer.

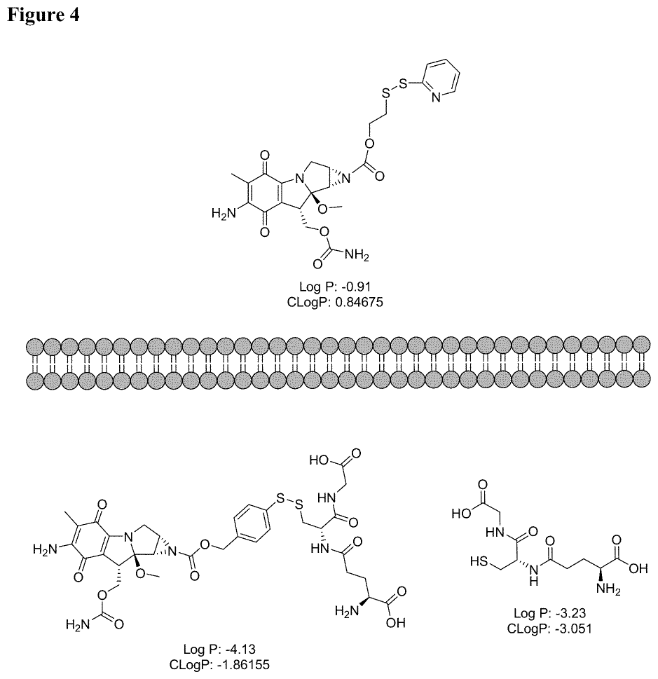

Drug leakage may be minimized by conjugating the drug with glutathione, effectively “trapping” the drug within the interior of the liposome. In one embodiment, MMC prodrug can be prepared with an activated disulfide. As the MMC prodrug crosses the lipid bilayer, the thiol moiety of glutathione reacts with the prodrug generating a glutathione-MMC prodrug conjugate. FIG. 4 illustrates the glutathione trapping of a MMC prodrug within the liposome. The glutathione-MMC prodrug conjugate has a slower propensity to leak from the liposome resulting in a more sustained and controlled release of the MMC prodrug from the liposome. Other thiol-containing compounds can also be used to entrap a MMC prodrug within the liposome, such as but not limited to, cysteine, homocysteine, cysteamine, mercaptoacetic acid, metcapto-succinic acid, thioglycolic acid, captopril, and 6-mercaptohexanoic acid.

Similarly, other therapeutic or diagnostic agents may be derivatized with glutathione and trapped within the liposome to affect sustained, controlled release of the drug from the liposome. Non-limiting examples of such agents include, but are not limited to, doxorubicin, paclitaxel, vinblastine, vincristiine, mertansine, irinotecan and epothilones. Further, therapeutic and/or diagnostic agents that contain a reactive double bond (one that is susceptible toward a “Michael” addition of the thiol, such as a maleimide) can be trapped via remote loading. For example, the schematic below shows the reaction of glutathione with a maleimide-modified BODIPY dye.

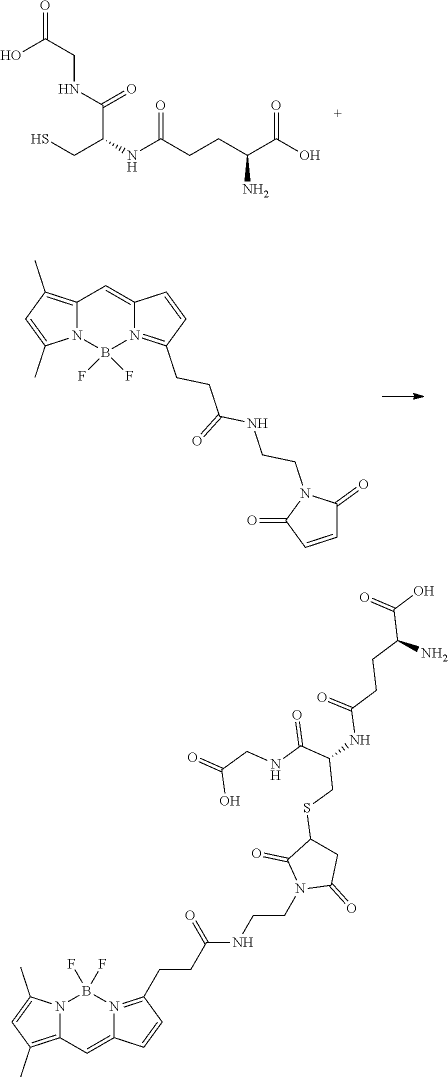

In one embodiment, the invention provides a method for preparing MMC prodrug liposomal formulations. The method includes: a) forming a first liposome having a lipid bilayer including a phosphatidylcholine lipid, a PEG-lipid and a sterol, wherein the lipid bilayer encapsulates an interior compartment comprising an aqueous solution and a thiol-containing compound, such as glutathione; and b) loading the first liposome with a MMC prodrug or a pharmaceutically-acceptable salt thereof, to form a loaded liposome.

### D. Diagnostic Agents

The liposomes of the present invention may also contain diagnostic agents. A diagnostic agent used in the present invention can include any diagnostic agent known in the art, as provided, for example, in the following references: Armstrong et al., Diagnostic Imaging, 5th Ed., Blackwell Publishing (2004); Torchilin, V. P., Ed., Targeted Delivery of Imaging Agents, CRC Press (1995); Vallabhajosula, S., Molecular Imaging: Radiopharmaceuticals for PET and SPECT, Springer (2009). A diagnostic agent can be detected by a variety of ways, including as an agent providing and/or enhancing a detectable signal that includes, but is not limited to, gamma-emitting, radioactive, echogenic, optical, fluorescent, absorptive, magnetic, or tomography signals. Techniques for imaging the diagnostic agent can include, but are not limited to, single photon emission computed tomography (SPECT), magnetic resonance imaging (MRI), optical imaging, positron emission tomography (PET), computed tomography (CT), x-ray imaging, gamma ray imaging, and the like. The diagnostic agents can be associated with the therapeutic liposome in a variety of ways, including for example being embedded to or encapsulated in the liposome.

In some embodiments, a diagnostic agent can include chelators that bind to metal ions to be used for a variety of diagnostic imaging techniques. Exemplary chelators include, but are not limited to, ethylenediaminetetraacetic acid (EDTA), [4-(1,4,8, 11-tetraazacyclotetradec-1-yl) methyl]benzoic acid (CPTA), cyclohexanediaminetetraacetic acid (CDTA), ethylenebis(oxyethylenenitrilo)tetraacetic acid (EGTA), diethylenetriaminepentaacetic acid (DTPA), citric acid, hydroxyethyl ethylenediamine triacetic acid (HEDTA), iminodiacetic acid (IDA), triethylene tetraamine hexaacetic acid (TTHA), 1,4,7,10-tetraazacyclododecane-1,4,7,10-tetra (methylene phosphonic acid) (DOTP), 1,4,8,11-tetraazacyclododecane-1,4,8,11-tetraacetic acid (TETA), 1,4,7,10-tetraazacyclododecane-1,4,7,10-tetraacetic acid (DOTA), and derivatives thereof.

A radioisotope can be incorporated into some of the diagnostic agents described herein and can include radionuclides that emit gamma rays, positrons, beta and alpha particles, and X-rays. Suitable radionuclides include but are not limited to ²²⁵AC, ⁷²As, ²¹¹At, ¹¹B, ¹²⁸Ba, ²¹²Bi, ⁷⁵Br, ⁷⁷Br, ¹⁴C, ₁₀₉Cd, ⁶²Cu, ⁶⁴Cu, ⁶⁷Cu, ¹⁸F, ⁶⁷Ga, ⁶⁸Ga, ³H, ¹²³I, ¹²⁵I, ¹³⁰I, ¹³¹I, ¹¹¹In, ¹⁷⁷Lu, ¹³N, ¹⁵O, ³²P , ³³P, ²¹²Pb, ¹⁰³Pd, ¹⁸⁶Re, ¹⁸⁸Re, ⁴⁷Sc, ¹⁵³Sm, ⁸⁹Sr, ^{99m}Tc, ⁸⁸Y, and ⁹⁰Y. In certain embodiments, radioactive agents can include ¹¹¹In-DTPA, ^{99m}TC(CO)₃-DTPA, ^{99m}Tc(CO)₃-ENPy2, ^{62/64/67}Cu-TETA, ^{99m}Tc(CO)₃-IDA, and ^{99m}Tc(CO)₃triamines (cyclic or linear). In other embodiments, the agents can include DOTA and its various analogs with ¹¹¹In, ¹⁷⁷Lu, ¹⁵³Sm, ^{88/90}Y, ^{62/64/67}Cu, or ^{67/68}Ga. In some embodiments, the liposomes can be radiolabeled, for example, by incorporation of lipids attached to chelators, such as DTPA-lipid, as provided in the following references: Phillips et al., Wiley Interdisciplinary Reviews: Nanomedicine and Nanobiotechnology, 1(1): 69-83 (2008); Torchilin, V. P. & Weissig, V., Eds. Liposomes 2nd Ed.: Oxford Univ. Press (2003); Elbayoumi, T. A. & Torchilin, V. P., Eur. J. Nucl. Med. Mol. Imaging 33:1196-1205 (2006); Mougin-Degraef, M. et al., Int'l J. Pharmaceutics 344:110-117 (2007).

In other embodiments, the diagnostic agents can include optical agents such as fluorescent agents, phosphorescent agents, chemiluminescent agents, and the like. Numerous agents (e.g., dyes, probes, labels, or indicators) are known in the art and can be used in the present invention. (See, e.g., Invitrogen, The Handbook—A Guide to Fluorescent Probes and Labeling Technologies, 10th Ed. (2005)). Fluorescent agents can include a variety of organic and/or inorganic small molecules or a variety of fluorescent proteins and derivatives thereof. For example, fluorescent agents can include, but are not limited to, cyanines, phthalocyanines, porphyrins, indocyanines, rhodamines, phenoxazines, phenylxanthenes, phenothiazines, phenoselenazines, fluoresceins, benzoporphyrins, squaraines, dipyrrolo pyrimidones, tetracenes, quinolines, pyrazines, corrins, croconiums, acridones, phenanthridines, rhodamines, acridines, anthraquinones, chalcogenopyrylium analogues, chlorins, naphthalocyanines, methine dyes, indolenium dyes, azo compounds, azulenes, azaazulenes, triphenyl methane dyes, indoles, benzoindoles, indocarbocyanines, benzoindocarbocyanines, and BODIPY™ derivatives having the general structure of 4,4-difluoro-4-bora-3a,4a-diaza-s-indacene, and/or conjugates and/or derivatives of any of these. Other agents that can be used include, but are not limited to, for example, fluorescein, fluorescein-polyaspartic acid conjugates, fluorescein-polyglutamic acid conjugates, fluorescein-polyarginine conjugates, indocyanine green, indocyanine-dodecaaspartic acid conjugates, indocyanine-polyaspartic acid conjugates, isosulfan blue, indole disulfonates, benzoindole disulfonate, bis(ethylcarboxymethyl)indocyanine, bis(pentylcarboxymethyl)indocyanine, polyhydroxyindole sulfonates, polyhydroxybenzoindole sulfonate, rigid heteroatomic indole sulfonate, indocyaninebispropanoic acid, indocyaninebishexanoic acid, 3,6-dicyano-2,5-[(N,N,N′,N′-tetrakis (carboxymethyl)amino]pyrazine, 3,6-[(N,N,N′,N′-tetrakis(2-hydroxyethyl) amino]pyrazine-2,5 -dicarboxylic acid, 3,6-bis(N-azatedino)pyrazine-2,5-dicarboxylic acid, 3,6-bis(N-morpholino)pyrazine-2,5 -dicarboxylic acid, 3,6-bis(N-piperazino) pyrazine-2,5 -dicarboxylic acid, 3,6-bis(N-thiomorpholino)pyrazine-2,5 -dicarboxylic acid, 3,6-bis(N-thiomorpholino)pyrazine-2,5 -dicarboxylic acid S-oxide, 2,5 -dicyano-3,6-bis (N-thiomorpholino)pyrazine S,S-dioxide, indocarbocyaninetetrasulfonate, chloroindocarbocyanine, and 3,6-diaminopyrazine-2,5 -dicarboxylic acid.

One of ordinary skill in the art will appreciate that particular optical agents for use with the invention can depend on the wavelength used for excitation, depth underneath skin tissue, and other factors generally well known in the art. For example, optimal absorption or excitation maxima for the optical agents can vary depending on the agent employed, but in general, the optical agents of the present invention will absorb or be excited by light in the ultraviolet (UV), visible, or infrared (IR) range of the electromagnetic spectrum. For imaging, dyes that absorb and emit in the near-IR (approximately 700-900 nm, e.g., indocyanines) are preferred. For topical visualization using an endoscopic method, any dyes absorbing in the visible range are suitable.

In some embodiments, the non-ionizing radiation employed in the process of the present invention can range in wavelength from about 350 nm to about 1200 nm. In one exemplary embodiment, the fluorescent agent can be excited by light having a wavelength in the blue range of the visible portion of the electromagnetic spectrum (from about 430 nm to about 500 nm) and emits at a wavelength in the green range of the visible portion of the electromagnetic spectrum (from about 520 nm to about 565 nm). For example, fluorescent dyes can be excited with light with a wavelength of about 488 nm and have an emission wavelength of about 520 nm. As another example, 3,6-diaminopyrazine-2,5-dicarboxylic acid can be excited with light having a wavelength of about 470 nm and fluoresces at a wavelength of about 532 nm. In another embodiment, the excitation and emission wavelengths of the optical agent may fall in the near-infrared range of the electromagnetic spectrum. For example, indocyanine dyes, such as indocyanine green, can be excited with light with a wavelength of about 780 nm and have an emission wavelength of about 830 nm.

In yet other embodiments, the diagnostic agents can include, but are not limited to, magnetic resonance (MR) and x-ray contrast agents that are generally well known in the art, including, for example, iodine-based x-ray contrast agents, superparamagnetic iron oxide (SPIO), complexes of gadolinium or manganese, and the like. (See, e.g., Armstrong et al., Diagnostic Imaging, 5th Ed., Blackwell Publishing (2004)). In some embodiments, a diagnostic agent can include a MR imaging agent. Exemplary MR agents include, but are not limited to, paramagnetic agents, superparamagnetic agents, and the like. Exemplary paramagnetic agents can include, but are not limited to, gadopentetic acid, gadoteric acid, gadodiamide, gadolinium, gadoteridol, mangafodipir, gadoversetamide, ferric ammonium citrate, gadobenic acid, gadobutrol, or gadoxetic acid. Superparamagnetic agents can include, but are not limited to, superparamagnetic iron oxide and ferristene. In certain embodiments, the diagnostic agents can include x-ray contrast agents as provided, for example, in the following references: H. S Thomsen, R. N. Muller and R. F. Mattrey, Eds., Trends in Contrast Media, (Berlin: Springer-Verlag, 1999); P. Dawson, D. Cosgrove and R. Grainger, Eds., Textbook of Contrast Media (ISIS Medical Media 1999); Torchilin, V. P., Curr. Pharm. Biotech. 1:183-215 (2000); Bogdanov, A. A. et al., Adv. Drug Del. Rev. 37:279-293 (1999); Sachse, A. et al., Investigative Radiology 32(1):44-50 (1997). Examples of x-ray contrast agents include, without limitation, iopamidol, iomeprol, iohexol, iopentol, iopromide, iosimide, ioversol, iotrolan, iotasul, iodixanol, iodecimol, ioglucamide, ioglunide, iogulamide, iosarcol, ioxilan, iopamiron, metrizamide, iobitridol and iosimenol. In certain embodiments, the x-ray contrast agents can include iopamidol, iomeprol, iopromide, iohexol, iopentol, ioversol, iobitridol, iodixanol, iotrolan, and iosimenol.

### E. Targeting Agents

In some cases, liposome accumulation at a target site may be due to the enhanced permeability and retention characteristics of certain tissues such as cancer tissues. Accumulation in such a manner often results in part because of liposome size and may not require special targeting functionality. In other cases, the liposomes of the present invention can also include a targeting agent. Generally, the targeting agents of the present invention can associate with any target of interest, such as a target associated with an organ, tissues, cell, extracellular matrix, or intracellular region. In certain embodiments, a target can be associated with a particular disease state, such as a cancerous condition. In some embodiments, the targeting component can be specific to only one target, such as a receptor. Suitable targets can include, but are not limited to, a nucleic acid, such as a DNA, RNA, or modified derivatives thereof. Suitable targets can also include, but are not limited to, a protein, such as an extracellular protein, a receptor, a cell surface receptor, a tumor-marker, a transmembrane protein, an enzyme, or an antibody. Suitable targets can include a carbohydrate, such as a monosaccharide, disaccharide, or polysaccharide that can be, for example, present on the surface of a cell.

In certain embodiments, a targeting agent can include a target ligand (e.g., an RGD-containing peptide), a small molecule mimic of a target ligand (e.g., a peptide mimetic ligand), or an antibody or antibody fragment specific for a particular target. In some embodiments, a targeting agent can further include folic acid derivatives, B-12 derivatives, integrin RGD peptides, NGR derivatives, somatostatin derivatives or peptides that bind to the somatostatin receptor, e.g., octreotide and octreotate, and the like. The targeting agents of the present invention can also include an aptamer. Aptamers can be designed to associate with or bind to a target of interest. Aptamers can be comprised of, for example, DNA, RNA, and/or peptides, and certain aspects of aptamers are well known in the art. (See. e.g., Klussman, S., Ed., The Aptamer Handbook, Wiley-VCH (2006); Nissenbaum, E. T., Trends in Biotech. 26(8): 442-449 (2008)).

### F. Methods for Preparing Liposomal MMC Prodrugs

In one embodiment, the invention provides methods for preparing liposomal MMC prodrugs. Liposomes can be prepared and loaded with MMC prodrugs using a number of techniques that are known to those of skill in the art. Lipid vesicles can be prepared, for example, by hydrating a dried lipid film (prepared via evaporation of a mixture of the lipid and an organic solvent in a suitable vessel) with water or an aqueous buffer. Hydration of lipid films typically results in a suspension of multilamellar vesicles (MLVs). Alternatively, MLVs can be formed by diluting a solution of a lipid in a suitable solvent, such as a C₁₋₄ alkanol, with water or an aqueous buffer. Unilamellar vesicles can be formed from MLVs via sonication or extrusion through membranes of defined pore sizes. Encapsulation of a MMC prodrug can be conducted by including the drug in the aqueous solution used for film hydration or lipid dilution during MLV formation. MMC prodrugs can also be encapsulated in pre-formed vesicles using “remote loading” techniques. Remote loading includes the establishment of a pH- or ion-gradient on either side of the vesicle membrane, which drives the MMC prodrug from the exterior solution to the interior of the vesicle.

Accordingly, some embodiments of the present invention provide a method for preparing a liposomal MMC prodrug including: a) forming a first liposome having a lipid bilayer including a PC lipid, PEG-lipid, and a sterol, wherein the lipid bilayer encapsulates an interior compartment containing an aqueous solution; and b) loading the first liposome with a MMC prodrug, or a pharmaceutically-acceptable salt thereof, to form a loaded liposome.

The MMC prodrug and lipids used in the methods of the invention are generally as described above. However, the route to the liposomal MMC prodrug will depend in part on the identity of the specific MMC prodrug and lipids and the quantities and combinations that are used. For example, the MMC prodrug can be encapsulated in vesicles at various stages of liposome preparation. In some embodiments, the first liposome is formed such that the lipid bilayer comprises DSPC and cholesterol, and the DSPC:cholesterol ratio is about 55:40 (mol:mol). In another embodiments, the first liposome is formed such that the lipid bilayer comprises DSPC and cholesterol, and the DSPC:cholesterol ratio is about 70:30 (mol:mol). In some embodiments, the first liposome is formed such that the lipid bilayer comprises DSPC, DSPE-PEG(2000), and cholesterol, and the DSPC:DSPE-PEG(2000):cholesterol ratio is about 55:40:5 (mol:mol). In some embodiments, the interior of the first liposome contains aqueous ammonium citrate buffer. Loading the first liposomes can include forming an aqueous solution containing the first liposome and the MMC prodrug or pharmaceutically-acceptable salt thereof under conditions sufficient to allow accumulation of the MMC prodrug in the interior compartment of the first liposome.

Loading conditions generally include a higher ammonium citrate concentration in the interior of the first liposome than in the exterior aqueous solution. In some embodiments, the loading step is conducted at a temperature above the gel-to-fluid phase transition temperature (Tₘ) of one or more of the lipid components in the liposomes. The loading can be conducted, for example, at about 50, about 55, about 60, about 65, or at about 70° C. In some embodiments, the loading step is conducted at a temperature of from about 50° C. to about 70° C. Loading can be conducted using any suitable amount of the MMC prodrug. In general, the MMC prodrug is used in an amount such that the ratio of the combined weight of the phosphatidylcholine, PEG-lipid, and the sterol in the liposome to the weight of the MMC prodrug is from about 1:0.01 to about 1:1. The ratio of the combined phosphatidylcholine/sterol to the weight of the MMC prodrug can be, for example, about 1:0.01, about 1:0.05, about 1:0.10, about 1:0.15, about 1:0.20, about 1:0.25, about 1:0.30, about 1:0.35, about 1:0.40, about 1:0.45, about 1:0.50, about 1:0.55, about 1:0.60, about 1:0.65, about 1:0.70, about 1:0.75, about 1:0.80, about 1:0.85, about 1:0.90, about 1:0.95, or about 1:1. In some embodiments, the loading step is conducted such that the ratio of the combined weight of the phosphatidylcholine and the sterol to the weight of the MMC prodrug is from about 1:0.01 to about 1:1. In some embodiments, the ratio of the combined weight of the phosphatidylcholine and the sterol to the weight of the MMC prodrug is from about 1:0.05 to about 1:0.5. In some embodiments, the ratio of the combined weight of the phosphatidylcholine and the sterol to the weight of the MMC prodrug is about 1:0.2. The loading step can be conducted for any amount of time that is sufficient to allow accumulation of the MMC prodrug in the liposome interior at a desired level.

In another embodiment, the MMC prodrug can be remotely loaded into the first liposome via an encapsulated thiol-containing compound, such as glutathione. Remote loading includes the establishment of a pH- or ion-gradient on either side of the vesicle membrane, which drives the MMC prodrug from the exterior solution to the interior of the vesicle. Alternatively, the MMC prodrug can be loaded into the first liposome via equilibrium passage through the lipid bilayer. The thiol-containing compound, such as glutathione reacts with the MMC prodrug to form a conjugate in the interior compartment of the liposome.

In one embodiment, the invention provides a method for preparing MMC prodrug liposomal formulations. The method includes: a) forming a first liposome having a lipid bilayer including a PC lipid, a PEG-lipid, and a sterol, wherein the lipid bilayer encapsulates an interior compartment comprising an aqueous solution and a thiol-containing compound, such as glutathione; and b) loading the first liposome with a MMC prodrug or a pharmaceutically-acceptable salt thereof to form a loaded liposome.

The PEG-lipid can also be incorporated into lipid vesicles at various stages of the liposome preparation. For example, MLVs containing a PEG-lipid can be prepared prior to loading with a MMC prodrug. Alternatively, a PEG-lipid can be inserted into a lipid bilayer after loading of a vesicle with a MMC prodrug. The PEG-lipid can be inserted into MLVs prior to extrusion of SUVs, or the PEG-lipid can be inserted into pre-formed SUVs.

Accordingly, some embodiments of the invention provide a method for preparing a liposomal MMC prodrug wherein the method includes: a) forming a first liposome having a lipid bilayer including a PC lipid and a sterol, wherein the lipid bilayer encapsulates an interior compartment comprising an aqueous solution; b) loading the first liposome with a MMC prodrug, or a pharmaceutically-acceptable salt thereof to form a loaded liposome; and c) forming a mixture containing the loaded liposome and a PEG-lipid under conditions sufficient to allow insertion of the PEG-lipid into the lipid bilayer.

In some embodiments, the insertion of the PEG-lipid is conducted at a temperature of from about 35 to about 70° C. The insertion of the PEG-lipid can be conducted, for example, at about 35° C., about 40° C., about 45° C., about 50° C., about 55° C., about 60° C., about 65° C., or about 70° C. In some embodiments, insertion of the PEG-lipid is conducted at a temperature of from about 50° C. to about 60° C. In a further embodiment, insertion of the PEG-lipid is conducted at a temperature of from about 50° C. to about 55° C.

In some embodiments, the insertion of the PEG-lipid is conducted for about 15 to about 75 min. In another embodiment, the insertion of the PEG-lipid is conducted for about 30 to about 60 min. The insertion of the PEG-lipid can be conducted, for example, for about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, or about 60 min. In some embodiments, insertion of the PEG-lipid is conducted for about 50 to about 60 min. In a further embodiment, insertion of the PEG-lipid is conducted for about 50 to about 55 min.

In some embodiments, the insertion of the PEG-lipid is conducted at a temperature of from about 35 to about 70° C. for about 15 to about 70 min. The insertion of the PEG-lipid can be conducted at a temperature of from about 50 to about 70° C. for about 30 to about 60 min, from about 50 to about 60° C. for about 30 to about 60 min, or from about 50 to about 55° C. for about 30 to about 60 min. In a further embodiment, insertion of the PEG-lipid is conducted at a temperature of about 55° C. for about 60 min.

Insertion can be conducted using any suitable amount of the PEG-lipid. In general, the PEG-lipid is used in an amount such that the ratio of the combined number of moles of the phosphatidylcholine and the sterol to the number of moles of the PEG-lipid is from about 1000:1 to about 20:1. The molar ratio of the combined phosphatidylcholine/sterol to PEG lipid can be, for example, about 1000:1, about 950:1, about 900:1, about 850:1, about 800:1, about 750:1, about 700:1, about 650:1, about 600:1, about 550:1, about 500:1, about 450:1, about 400:1, about 350:1, about 300:1, about 250:1, about 200:1, about 150:1, about 100:1, about 50:1, or about 20:1. In some embodiments, the ratio of the combined phosphatidylcholine and sterol to the PEG-lipid is from about 100:1 to about 20:1 (mol:mol). In some embodiments, the ratio of the combined phosphatidylcholine and sterol to the PEG-lipid is from about 35:1 to about 25:1 (mol:mol). In some embodiments, the ratio of the combined phosphatidylcholine and sterol to the PEG-lipid is about 33:1 (mol:mol). In some embodiments, the ratio of the combined phosphatidylcholine and sterol to the PEG-lipid is about 27:1 (mol:mol).

A number of additional preparative techniques known to those of skill in the art can be included in the methods of the invention. Liposomes can be exchanged into various buffers by techniques including dialysis, size exclusion chromatography, diafiltration, and ultrafiltration. Buffer exchange can be used to remove unencapsulated MMC prodrugs and other unwanted soluble materials from the compositions. Aqueous buffers and certain organic solvents can be removed from the liposomes. In some embodiments, the methods of the invention include exchanging the liposomal MMC prodrug from the mixture in step c) to an aqueous solution that is substantially free of unencapsulated MMC prodrug and uninserted PEG-lipid. In some embodiments, the methods include lyophilizing the liposomal MMC prodrug.

### G. Methods of Treating Cancer

In another embodiment, the invention provides a method of treating cancer. The method includes administering to a subject in need thereof a composition containing a liposomal MMC prodrug as described above. In therapeutic use for the treatment of cancer, the liposome compositions of the present invention can be administered such that the initial dosage of the MMC prodrug ranges from about 0.001 mg/kg to about 500 mg/kg daily. A daily dose of about 0.01 to about 500 mg/kg, or about 0.1 to about 200 mg/kg, or about 1 to about 100 mg/kg, or about 10 to about 50 mg/kg, or about 15 mg/kg, or about 10 mg/kg, or about 5 mg/kg, or about 2.5 mg/kg, or about 1 mg/kg can be used. Further, a daily dose of 3,6, 12, 24, 48, 80, 120, 160, 190, 225, 270, and 320 mg/m² can be used.

The dosages may be varied depending upon the requirements of the patient, the type and severity of the cancer being treated, and the liposome composition being employed. For example, dosages can be empirically determined considering the type and stage of cancer diagnosed in a particular patient. The dose administered to a patient should be sufficient to affect a beneficial therapeutic response in the patient over time. In another embodiment, the dose administered to a patient should be sufficient to treat the patient. The size of the dose will also be determined by the existence, nature, and extent of any adverse side-effects that accompany the administration of a particular liposome composition in a particular patient. Determination of the proper dosage for a particular situation is within the skill of the practitioner. Generally, treatment is initiated with smaller dosages which are less than the optimum dose of the liposome composition. Thereafter, the dosage is increased by small increments until the optimum effect under the circumstances is reached. For convenience, the total daily dosage may be divided and administered in portions during the day, if desired.

The methods described herein apply especially to solid tumor cancers (solid tumors), which are cancers of organs and tissue (as opposed to hematological malignancies), and ideally epithelial cancers. Examples of solid tumor cancers include bladder cancer, breast cancer, cervical cancer, colorectal cancer (CRC), esophageal cancer, gastric cancer, head and neck cancer, hepatocellular cancer, lung cancer, melanoma, neuroendocrine cancer, ovarian cancer, pancreatic cancer, prostate cancer, renal cancer, and thymus cancer. In one group of embodiments, the solid tumor cancer suitable for treatment according to the methods of the invention are selected from CRC, breast cancer, and prostate cancer. In another group of embodiments, the methods of the invention apply to treatment of hematological malignancies, including for example multiple myeloma, T-cell lymphoma, B-cell lymphoma, Hodgkins disease, non-Hodgkins lymphoma, acute myeloid leukemia, and chronic myelogenous leukemia.

The compositions in the methods of the invention may be administered alone or in combination with other therapeutic agents. The additional agents can be anticancer agents belonging to several classes of drugs such as, but not limited to, cytotoxic agents, VEGF-inhibitors, tyrosine kinase inhibitors, monoclonal antibodies, and immunotherapies. Examples of such agents include, but are not limited to, doxorubicin, cisplatin, oxaliplatin, carboplatin, 5-fluorouracil, gemcitabine (anti-metabolite), ramucirumab (VEGF 2 inhibitor), bevacizumab (VEGF inhibitor), trastuzumab (monoclonal antibody HER2 inhibitor), afatinib (EGFR tyrosine kinase inhibitor), and others. Additional anti-cancer agents can include, but are not limited to, 20-epi-1,25 dihydroxyvitamin D3,4-ipomeanol, 5-ethynyluracil, 9-dihydrotaxol, abiraterone, acivicin, aclarubicin, acodazole hydrochloride, acronine, acylfulvene, adecypenol, adozelesin, aldesleukin, all-tk antagonists, altretamine, ambamustine, ambomycin, ametantrone acetate, amidox, amifostine, aminoglutethimide, aminolevulinic acid, amrubicin, amsacrine, anagrelide, anastrozole, andrographolide, angiogenesis inhibitors, antagonist D, antagonist G, antarelix, anthramycin, anti-dorsalizing morphogenetic protein-1, antiestrogen, antineoplaston, antisense oligonucleotides, aphidicolin glycinate, apoptosis gene modulators, apoptosis regulators, apurinic acid, ARA-CDP-DL-PTBA, arginine deaminase, asparaginase, asperlin, asulacrine, atamestane, atrimustine, axinastatin 1, axinastatin 2, axinastatin 3, azacitidine, azasetron, azatoxin, azatyrosine, azetepa, azotomycin, baccatin III derivatives, balanol, batimastat, benzochlorins, benzodepa, benzoylstaurosporine, beta lactam derivatives, beta-alethine, betaclamycin B, betulinic acid, BFGF inhibitor, bicalutamide, bisantrene, bisantrene hydrochloride, bisaziridinylspermine, bisnafide, bisnafide dimesylate, bistratene A, bizelesin, bleomycin, bleomycin sulfate, BRC/ABL antagonists, breflate, brequinar sodium, bropirimine, budotitane, busulfan, buthionine sulfoximine, cactinomycin, calcipotriol, calphostin C, calusterone, camptothecin derivatives, canarypox IL-2, capecitabine, caracemide, carbetimer, carboplatin, carboxamide-amino-triazole, carboxyamidotriazole, carest M3, carmustine, cam 700, cartilage derived inhibitor, carubicin hydrochloride, carzelesin, casein kinase inhibitors, castanospermine, cecropin B, cedefingol, cetrorelix, chlorambucil, chlorins, chloroquinoxaline sulfonamide, cicaprost, cirolemycin, cisplatin, cis-porphyrin, cladribine, clomifene analogs, clotrimazole, collismycin A, collismycin B, combretastatin A4, combretastatin analog, conagenin, crambescidin 816, crisnatol, crisnatol mesylate, cryptophycin 8, cryptophycin A derivatives, curacin A, cyclopentanthraquinones, cyclophosphamide, cycloplatam, cypemycin, cytarabine, cytarabine ocfosfate, cytolytic factor, cytostatin, dacarbazine, dacliximab, dactinomycin, daunorubicin hydrochloride, decitabine, dehydrodidemnin B, deslorelin, dexifosfamide, dexormaplatin, dexrazoxane, dexverapamil, dezaguanine, dezaguanine mesylate, diaziquone, didemnin B, didox, diethylnorspermine, dihydro-5 -azacytidine, dioxamycin, diphenyl spiromustine, docetaxel, docosanol, dolasetron, doxifluridine, doxorubicin, doxorubicin hydrochloride, droloxifene, droloxifene citrate, dromostanolone propionate, dronabinol, duazomycin, duocarmycin SA, ebselen, ecomustine, edatrexate, edelfosine, edrecolomab, eflomithine, eflomithine hydrochloride, elemene, elsamitrucin, emitefur, enloplatin, enpromate, epipropidine, epirubicin, epirubicin hydrochloride, epristeride, erbulozole, erythrocyte gene therapy vector system, esorubicin hydrochloride, estramustine, estramustine analog, estramustine phosphate sodium, estrogen agonists, estrogen antagonists, etanidazole, etoposide, etoposide phosphate, etoprine, exemestane, fadrozole, fadrozole hydrochloride, fazarabine, fenretinide, filgrastim, finasteride, flavopiridol, flezelastine, floxuridine, fluasterone, fludarabine, fludarabine phosphate, fluorodaunorunicin hydrochloride, fluorouracil, fluorocitabine, forfenimex, formestane, fosquidone, fostriecin, fostriecin sodium, fotemustine, gadolinium texaphyrin, gallium nitrate, galocitabine, ganirelix, gelatinase inhibitors, gemcitabine, gemcitabine hydrochloride, glutathione inhibitors, hepsulfam, heregulin, hexamethylene bisacetamide, hydroxyurea, hypericin, ibandronic acid, idarubicin, idarubicin hydrochloride, idoxifene, idramantone, ifosfamide, ilmofosine, ilomastat, imidazoacridones, imiquimod, immunostimulant peptides, insulin-like growth factor-1 receptor inhibitor, interferon agonists, interferon alpha-2A, interferon alpha-2B, interferon alpha-N1, interferon alpha-N3, interferon beta-IA, interferon gamma-M, interferons, interleukins, iobenguane, iododoxorubicin, iproplatin, irinotecan, irinotecan hydrochloride, iroplact, irsogladine, isobengazole, isohomohalicondrin B, itasetron, jasplakinolide, kahalalide F, lamellarin-N triacetate, lanreotide, lanreotide acetate, leinamycin, lenograstim, lentinan sulfate, leptolstatin, letrozole, leukemia inhibiting factor, leukocyte alpha interferon, leuprolide acetate, leuprolide/estrogen/progesterone, leuprorelin, levamisole, liarozole, liarozole hydrochloride, linear polyamine analog, lipophilic disaccharide peptide, lipophilic platinum compounds, lissoclinamide 7, lobaplatin, lombricine, lometrexol, lometrexol sodium, lomustine, lonidamine, losoxantrone, losoxantrone hydrochloride, lovastatin, loxoribine, lurtotecan, lutetium texaphyrin, lysofylline, lytic peptides, maitansine, mannostatin A, marimastat, masoprocol, maspin, matrilysin inhibitors, matrix metalloproteinase inhibitors, maytansine, mechlorethamine hydrochloride, megestrol acetate, melengestrol acetate, melphalan, menogaril, merbarone, mercaptopurine, meterelin, methioninase, methotrexate, methotrexate sodium, metoclopramide, metoprine, meturedepa, microalgal protein kinase C inhibitors, MIF inhibitor, mifepristone, miltefosine, mirimostim, mismatched double stranded RNA, mitindomide, mitocarcin, mitocromin, mitogillin, mitoguazone, mitolactol, mitomalcin, mitomycin, mitomycin analogs, mitonafide, mitosper, mitotane, mitotoxin fibroblast growth factor-saporin, mitoxantrone, mitoxantrone hydrochloride, mofarotene, molgramostim, monoclonal antibody, human chorionic gonadotrophin, monophosphoryl lipid a/myobacterium cell wall SK, mopidamol, multiple drug resistance gene inhibitor, multiple tumor suppressor 1-based therapy, mustard anticancer agent, mycaperoxide B, mycobacterial cell wall extract, mycophenolic acid, myriaporone, n-acetyldinaline, nafarelin, nagrestip, naloxone/pentazocine, napavin, naphterpin, nartograstim, nedaplatin, nemorubicin, neridronic acid, neutral endopeptidase, nilutamide, nisamycin, nitric oxide modulators, nitroxide antioxidant, nitrullyn, nocodazole, nogalamycin, n-substituted benzamides, O6-benzylguanine, octreotide, okicenone, oligonucleotides, onapristone, ondansetron, oracin, oral cytokine inducer, ormaplatin, osaterone, oxaliplatin, oxaunomycin, oxisuran, paclitaxel, paclitaxel analogs, paclitaxel derivatives, palauamine, palmitoylrhizoxin, pamidronic acid, panaxytriol, panomifene, parabactin, pazelliptine, pegaspargase, peldesine, peliomycin, pentamustine, pentosan polysulfate sodium, pentostatin, pentrozole, peplomycin sulfate, perflubron, perfosfamide, perillyl alcohol, phenazinomycin, phenylacetate, phosphatase inhibitors, picibanil, pilocarpine hydrochloride, pipobroman, piposulfan, pirarubicin, piritrexim, piroxantrone hydrochloride, placetin A, placetin B, plasminogen activator inhibitor, platinum complex, platinum compounds, platinum-triamine complex, plicamycin, plomestane, porfimer sodium, porfiromycin, prednimustine, procarbazine hydrochloride, propyl bis-acridone, prostaglandin J2, prostatic carcinoma antiandrogen, proteasome inhibitors, protein A-based immune modulator, protein kinase C inhibitor, protein tyrosine phosphatase inhibitors, purine nucleoside phosphorylase inhibitors, puromycin, puromycin hydrochloride, purpurins, pyrazofurin, pyrazoloacridine, pyridoxylated hemoglobin polyoxyethylene conjugate, RAF antagonists, raltitrexed, ramosetron, RAS farnesyl protein transferase inhibitors, RAS inhibitors, RAS-GAP inhibitor, retelliptine demethylated, rhenium RE 186 etidronate, rhizoxin, riboprine, ribozymes, RII retinamide, RNAi, rogletimide, rohitukine, romurtide, roquinimex, rubiginone B1, ruboxyl, safingol, safingol hydrochloride, saintopin, sarcnu, sarcophytol A, sargramostim, SDI 1 mimetics, semustine, senescence derived inhibitor 1, sense oligonucleotides, signal transduction inhibitors, signal transduction modulators, simtrazene, single chain antigen binding protein, sizofuran, sobuzoxane, sodium borocaptate, sodium phenylacetate, solverol, somatomedin binding protein, sonermin, sparfosate sodium, sparfosic acid, sparsomycin, spicamycin D, spirogermanium hydrochloride, spiromustine, spiroplatin, splenopentin, spongistatin 1, squalamine, stem cell inhibitor, stem-cell division inhibitors, stipiamide, streptonigrin, streptozocin, stromelysin inhibitors, sulfinosine, sulofenur, superactive vasoactive intestinal peptide antagonist, suradista, suramin, swainsonine, synthetic glycosaminoglycans, talisomycin, tallimustine, tamoxifen methiodide, tauromustine, tazarotene, tecogalan sodium, tegafur, tellurapyrylium, telomerase inhibitors, teloxantrone hydrochloride, temoporfin, temozolomide, teniposide, teroxirone, testolactone, tetrachlorodecaoxide, tetrazomine, thaliblastine, thalidomide, thiamiprine, thiocoraline, thioguanine, thiotepa, thrombopoietin, thrombopoietin mimetic, thymalfasin, thymopoietin receptor agonist, thymotrinan, thyroid stimulating hormone, tiazofurin, tin ethyl etiopurpurin, tirapazamine, titanocene dichloride, topotecan hydrochloride, topsentin, toremifene, toremifene citrate, totipotent stem cell factor, translation inhibitors, trestolone acetate, tretinoin, triacetyluridine, triciribine, triciribine phosphate, trimetrexate, trimetrexate glucuronate, triptorelin, tropisetron, tubulozole hydrochloride, turosteride, tyrosine kinase inhibitors, tyrphostins, UBC inhibitors, ubenimex, uracil mustard, uredepa, urogenital sinus-derived growth inhibitory factor, urokinase receptor antagonists, vapreotide, variolin B, velaresol, veramine, verdins, verteporfin, vinblastine sulfate, vincristine sulfate, vindesine, vindesine sulfate, vinepidine sulfate, vinglycinate sulfate, vinleurosine sulfate, vinorelbine, vinorelbine tartrate, vinrosidine sulfate, vinxaltine, vinzolidine sulfate, vitaxin, vorozole, zanoterone, zeniplatin, zilascorb, zinostatin, zinostatin stimalamer, and zorubicin hydrochloride.

### H. In Vivo Pharmacokinetics Properties of the Liposomal Compositions

The liposomal composition disclosed herein may be formulated for oral, intravenous, intramuscular, intraperitoneal, or rectal delivery. Bioavailability is often assessed by comparing standard pharmacokinetic (pK) parameters such as Cₘₐₓ and AUC.

In one embodiment, the liposomal composition may produce a plasma pK profile characterized by Cₘₐₓ for MMC from about 1 μg/ml to about 1,000 μg/ml, from about 10 μg/ml to about 900 μg/ml, from about 100 μg/ml to about 800 μg/ml, or from about 200 μg/ml to about 500 μg/ml. In another embodiment, the Cₘₐₓ for MMC may be about 10, 25, 50, 75, 100, 200, 300, 400, 500, 600, 700, 800, 900 or 1000 μg/ml.

In one embodiment, the liposomal composition may produce a plasma pK profile characterized by AUCₐₗₗ for MMC from about 1,000 μg·hr/ml to about 25,000 μg·hr/ml, from about 5,000 μg·hr/ml to about 20,000 μg·hr/ml, or from about 10,000 μg·hr/ml to about 15,000 μg·hr/ml. In another embodiment, the AUC_{inf} for docetaxel may be about 1,000, 2,000, 3,000, 4,000, 5,000, 10,000, 15,000, 20,000 or 25,000 μg·hr/ml.

In an additional embodiment, the liposomal composition may produce a plasma pK profile characterized by t_{1/2} for MMC from about 5 hours to about 20 hours, from about 5 hours to about 15 hours, or from about 10 hours to about 15 hours. In another embodiment, the t_{1/2} for MMC from may be about 5, 6, 7, 8, 9, 10, 11, 12, 13, 14, 15, 16, 17, 18, 19 or 20 hours.

### IV. Examples

### Example 1. 2-(pyridin-2-yldisulfanyl)ethyl (6-(trifluoromethyl)-1H-benzo[d][1,2,3]triazol-1- yl) carbonate (MP-3819)

### Synthesis of MP-3819

MP-3819 was synthesized as follows:

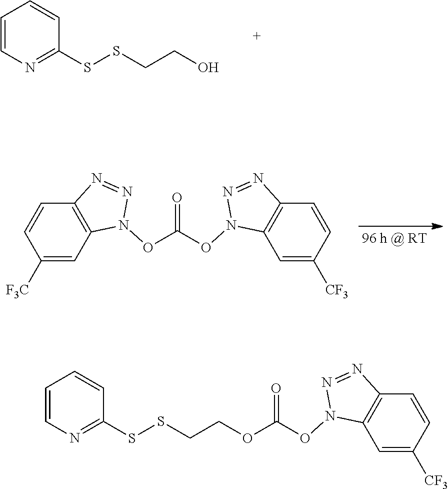 [SMILES: OCCSSc1ccccn1 ; O=C(On1nnc2ccc(C(F)(F)F)cc21)On1nnc2ccc(C(F)(F)F)cc21 ; O=C(OCCSSc1ccccn1)On1nnc2ccc(C(F)(F)F)cc21]

To bis(6-(trifluoromethyl)-1H-benzo[d][1,2,3]triazol-1-yl) carbonate (1.77 g, 4.11 mmol) in a 500-mL round-bottomed flask with stir bar was added acetonitrile (150 mL), and the mixture was stirred under nitrogen at room temperature (RT). A solution of 2-(pyridin-2-yldisulfanyl) ethanol (0.808 g, 4.31 mmol) dissolved in acetonitrile (10 mL) was dripped into the mixture over a 3 min period followed by a 15-mL rinse resulting in a clearing of the solution. The reaction was stirred under nitrogen at RT. The reaction was analyzed intermittently over the next couple of days by LC/MS showing the slow, increasing formation of product. After 96 h, the reaction was filtered by gravity through fluted paper into a 200-mL, round-bottomed flask, rinsed with acetonitrile, then rotavaped to a solid residue (2.53 g). To this solid was added 50 mL of 4% sodium bicarbonate and mixed to suspend the solid for some time. The solid was filtered by suction (sintered glass funnel), rinsed with water, then suctioned dry. The resulting solid was then dissolved with warm acetone, transferred to a 100-mL, round-bottomed flask and rotavaped to a solid again (1.72 g). Acetone (10 mL) was added to the solid and warmed gently to dissolve the solid. Diethyl ether (10 mL) was added, and the solution remained clear. The solution was then placed in the freezer overnight at which point crystals formed. The solution with crystals was permitted to warm to RT before collecting the solid by suction filtration and rinsing with methyl t-butyl ether. A second crop was obtained by cooling the solution in the freezer, warming to RT, collecting by suction filtration, and rinsing with methyl t-butyl ether. Both crops were combined and dried under high-vacuum. The combined crops provided 1.38 g (81% yield) of final product as an off-white solid.

LC/MS (5% -95% acetonitrile, 0.05% TFA): single peak at 4.5 min, [M+H]+=417.1 ¹H NMR (500 MHz, CDCl3) δ 8.47 (d, 1 H), 8.38 (d, 1 H), 8.33 (s, 1 H), 8.00 (dd, 1 H), 7.60 (m, 2 H), 7.11 (m, 1 H), 4.86 (t, 2 H), 3.28 (t, 2 H)

### Example 2. 2-((2-(piperidin-1-yl)ethyl)disulfanyl)ethyl(1aS,8S,8aR,8bS)-6-amino-8- (carbamoyloxy)methyl)-8a-methoxy-5-methyl-4,7-dioxo-1a,4,7,8,8a,8b- hexahydroazirino[2′,3′:3,4]pyrrolo[1,2-α]indole-1(2H)-carboxylate (MP-3827)

### Synthesis of MP-3827

MP-3827 was synthesized as described below:

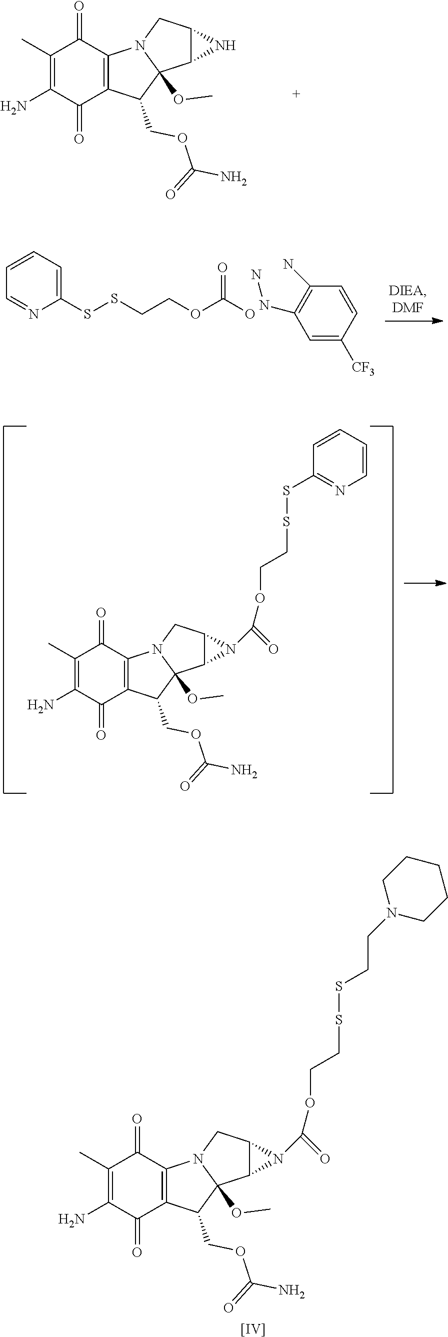

To a solution of MMC (95 mg, 0.28 mmol) and MP-3819 (118 mg, 0.28 mmol) in 2 mL DMF was added DIEA (0.050 mL, 0.28 mmol). The mixture was stirred at RT. At 1 h, the reaction was analyzed by LC/MS; the main peak showed the desired intermediate (m/z=548.2) at 6.34 minutes (Zorbax, SB-C18, StableBond, 4.6×150 mm, 5 u at 15 -95% acetonitrile/water with 0.05% TFA). 2-(piperidin-1-yl)ethane-1-thiol was then added, and the reaction was stirred for 1.5 h. Again by LC/MS, the main peak at 4.8 min showed the desired product (m/z=582.3). The reaction was diluted with ethyl acetate (10 mL), and the organic layer was washed with brine (15 mL) before concentrating to an oil. The residue was dissolved in 10% methanol/dichloromethane (1 mL), purified over Teledyne Silica Gold (20 g), and equilibrated with 3 CV of 10% methanol/dichloromethane. The clean, product-containing fractions were concentrated under reduced pressure. The material was redissolved in 1:1 water/acetonitrile (3 mL), frozen, and lyophilized to afford 55 mg (34% yield) of a light purple solid.

LC/MS (Zorbax, SB-C18, StableBond, 4.6×150 mm, 5 μm at 15-95% acetonitrile/water with 0.05% TFA): single peak, [M+H]+=582.3

### Conversion of MP-3827 to MMC

A 4 mg/mL solution of MP3827 was made in ethanol. 40 mg of cysteine HCl was dissolved in 10 mL deionized (DI) water (pH 2.1), and the pH was adjusted to 6.95 with sodium hydroxide solution. A 0.25 mL of the ethanol solution of MP3827 was added to 2.5 mL of the cysteine solution, and the reaction was monitored by HPLC. The solution was heated to 37° C. on a heating block. Periodically samples were removed for HPLC analysis, i.e., 25 μL sample was added into 0.45 mL methanol and then 1 mL DI water, and analyzed with HPLC assay at 360 nm. After 2 h, complete conversion was observed, generating 92% MMC (Area %).

### Preparation of Liposome (Ammonium Citrate)

14.14 g of ammonium citrate was weighed into a 250-ml volumetric flask and dissolved in deionized water (i.e., 250 mM ammonium citrate).

DSPC (1.49 g), cholesterol (0.53 g), and DSPE-PEG(2000) (0.48 g) were weighed into a 1 L round bottomed flask to which was added 10 mL of ethanol. This mixture was warmed to 70° C. to dissolve the lipids. 90 ml of 250 mM ammonium citrate was warmed to 70° C., and then added to the dissolved lipid components in ethanol at 70° C. and mixed with a rotary mixer at 70° C. for 15-30 min.

The crude vesicles were then extruded through double stacked 200 nm membranes (Lipex 100 mL extruder) at 70° C. to produce a uniform particle size of approximately 200 nm (nitrogen used, 5 times at 200 psi). The liposome mixture was then extruded through double stacked 100 nm membranes to produce a uniform particle size of approximately 100 nm. (nitrogen used, 5 times at 400 psi).

154.04 g of sucrose was dissolved in 1.5 L of Milli-Q water, pH=7.69. The 300 mM sucrose solution was then filtered through a 0.22 μm membrane filter to remove any particulates.

The liposomal ammonium citrate mixture was diafiltered against 10 volumes (1 L) sucrose solution (volume mean=101.9 nm, pH=6.68). Diafiltration was carried out using a KroFlo Research II, TFF System with a Hollow Fiber Filter Module (mPES/500 kD, Surface Area: 790 cm²).

### Remote Loading of MP-3827 into Liposome

125 mg of MP3827 was weighed into a serum bottle. To this was added 31 mL 0.3 M sucrose solution (filtered through 0.22 micron filter membrane), and the pH was adjusted to 5.8. 30 mL of the solution was heated to 60° C. in a water bath. 30 mL of DSPC:Cholesterol:DSPE-PEG(2000) 55:40:5 with encapsulated ammonium citrate were heated to 60° C. in a second serum bottle. At 60° C., the solution of MP-3827 was added all at once to the liposome and stirred at 60° C. for 20 min. The crude reaction was allowed to cool and then purified by passage through a Sepharose column (ca. 250 mL) using normal saline to elute the liposome. The liposome fraction was collected and concentrated using a 50 kDa centrifuge filter at 4000 rpm and at 4° C.

Assay: 25 μL of liposome was added to 475 μL methanol followed by 1 mL DI water. A clear purple solution was analyzed by HPLC at 360 nm and compared against a 4 mg/mL standard. The liposome contained 5.1 mg/mL MP3827 with a particle size of 102.3 nm (volume mean)

### Example 3. 4-(pyridin-2-yldisulfanyl)benzyl (6-(trifluoromethyl)-1H-benzo[d][1,2,3]triazol- 1-yl) carbonate (MP-3821)

### Synthesis of MP-3821

MP-3821 was synthesized as described below:

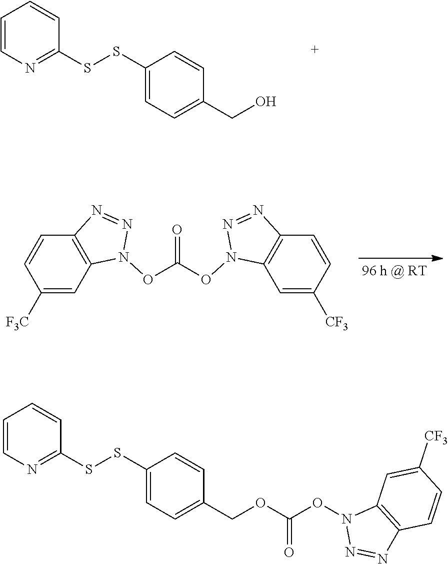 [SMILES: OCc1ccc(SSc2ccccn2)cc1 ; O=C(On1nnc2ccc(C(F)(F)F)cc21)On1nnc2ccc(C(F)(F)F)cc21 ; O=C(OCc1ccc(SSc2ccccn2)cc1)On1nnc2ccc(C(F)(F)F)cc21]

To bis(6-(trifluoromethyl)-1H-benzo[d][1,2,3]triazol-1-yl) carbonate (1.52 g, 3.52 mmol) in a 250-mL round-bottomed flask with stir bar was added acetonitrile (120 mL), and the mixture was stirred under argon at RT. To this mixture was dripped in (4-(pyridin-2-yldisulfanyl) phenyl)methanol (0.857 g, 3.44 mmol) dissolved in acetonitrile (5 mL) over five minutes and rinsed with 15 mL acetonitrile. After 88 h at RT, the reaction mixture was concentrated under reduced pressure to afford a solid residue (2.35 g). To this solid was added 30 mL of 4% NaHCO₃ (aq) with mixing to suspend the solid for some time. The material was collected by suction filtration and rinsed with water before suctioning dry. The solid was partially dissolved with warm acetone and diethyl ether. Additional acetone was added, and heating continued until all the material dissolved. Upon cooling, solid appeared, and the flask was kept in the freezer overnight. The solid was collected by suction filtration and rinsed with diethyl ether. After drying under high vacuum overnight, 0.879 g was obtained in the first crop.

LC/MS (5% -95% acetonitrile, 0.05% TFA): retention time=7.3 min., [M+H]+=479.1 ¹H NMR (500 MHz, CDCl₃) δ 8.47 (dd, 1 H), 8.32 (d, 2 H), 7.97 (dd, 1 H), 7.59 (m, 4 H), 7.45 (t, 2 H), 7.12 (m, 1 H), 5.52 (s, 2 H)

### Example 4 4-((2-(piperidin-1-yl)ethyl)disulfanyl)benzyl(1aS,8S,8aR,8bS)-6-amino-8- ((carbamoyloxy)methyl)-8a-methoxy-5-methyl-4,7-dioxo-1a,4,7,8,8a,8b- hexahydroazirino[2′,3′:3,4]pyrrolo[1,2-α]indole-1(2H)-carboxylate (MP-3832)

### Synthesis of MP-3832

MP-3832 was synthesized as described below:

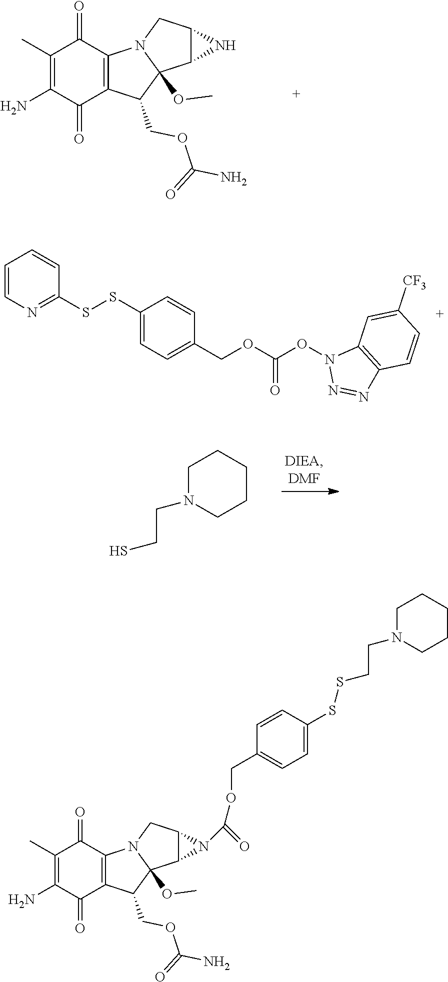 [SMILES: CO[C@@]12[C@H](COC(N)=O)C3=C(C(=O)C(C)=C(N)C3=O)N1C[C@@H]1N[C@@H]12 ; O=C(OCc1ccc(SSc2ccccn2)cc1)On1nnc2ccc(C(F)(F)F)cc21 ; SCCN1CCCCC1 ; CO[C@@]12[C@H](COC(N)=O)C3=C(C(=O)C(C)=C(N)C3=O)N1C[C@H]1[C@@H]2N1C(=O)OCc1ccc(SSCCN2CCCCC2)cc1]

To a stirring solution of MMC (199 mg, 0.596 mmol) and MP-3821 (285 mg, 0.596 mmol) in DMF (2 mL) was added DIEA (0.104 mL, 0.596 mmol). After stirring for 1 h, 2-(piperidin-1-yl)ethane-1-thiol (87 mg, 0.60 mmol) was added, and the reaction was continued overnight. The reaction was diluted with ethyl acetate, and the organic layer was washed with water several times. The organic layer was concentrated and reconstituted in dichloromethane for purification (4×1 mL portions) on a Buchi/Sunfire system using a gradient of 3-15% methanol in dichloromethane over 20 min at 165 mL/min (UV=210). The product-containing fractions were combined, concentrated, redissolved in acetonitrile/water, and lyophilized to give 11.9 mg of the desired product.

LC/MS (Zorbax, SB-C18, StableBond, 4.6×150 mm, 5 μm at 15-95% acetonitrile/water with 0.05% TFA): single peak at 6.2 min, [M+H]+=644.2

### Remote Loading of MP-3832 into Liposome

To 50 mg of MP-3832 was added 10 mL 0.3M sucrose (filtered through 0.22 micron membrane) in a glass scintillation vial. The pH was adjusted to 5. 12 mL of the MP-3832 solution was heated to 60° C. 12 mL of DSPC:Cholesterol:DSPE-PEG(2000) 55:40:5 with encapsulated ammonium citrate were heated to 60° C. in a 50-mL serum bottle. At 60° C., the solution of MP-3832 was added all at once to the liposome and stirred at 60° C. for 20 min. The crude reaction was allowed to cool and then purified by passage through a Sepharose column (ca. 200 mL) using normal saline to elute the liposome. The liposome fraction was collected and concentrated using a 50 kDa centrifuge filter at 4000 rpm and at 4° C.

25 μL sample was added into 475 μL methanol then diluted with 1 mL DI water, and analyzed by HPLC at 250 nm and compared against a 4 mg/mL standard. The liposome contained 3.4 mg/mL MP-3832 with a particle size of 101.1 nm (volume mean).

### Example 5. 2-((2-(4-methylpiperazin-1-yl)ethyl)disulfanyl)ethyl (1aS,8S,8aR,8bS)-6-amino- 8-((carbamoyloxy)methyl)-8a-methoxy-5-methyl-4,7-dioxo-1a,4,7,8,8a,8b- hexahydroazirino[2′,3′:3,4]pyrrolo[1,2-a]indole-1(2H)-carboxylate (MP-3854)

### Synthesis of MP-3854

MP-3854 was synthesized as described below:

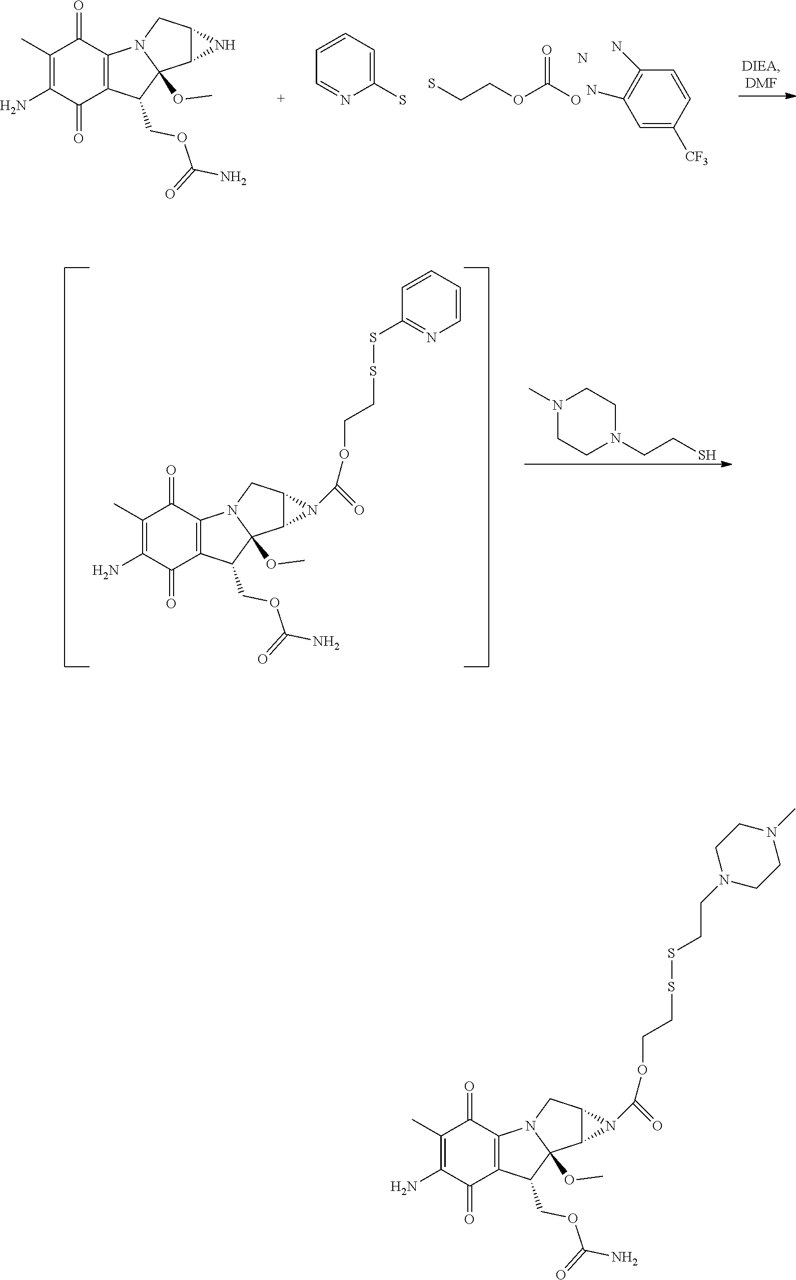

To a solution of MMC (300 mg, 0.897 mmol) and MP-3819 (374 mg, 0.897 mmol) in DMF (5 ml) was added DIEA (0.157 ml, 0.897 mmol). The mixture was stirred at RT. At 30 min., the reaction was checked by LC/MS (Zorbax, SB-C18, StableBond, 4.6×150 mm, 5 u at 15-60% acetonitrile/water with 0.05% TFA), which indicated approximately 75% of the desired intermediate (m/z=548.2) was formed. At this point, 2-(4-methylpiperazin-1-yl)ethanethiol (144 mg, 0.897 mmol) was added, and the reaction was stirred for 1.5 h. The reaction was then diluted with ethyl acetate (10 mL) and washed with saturated sodium chloride (25 ml). The aqueous layer was extracted with additional ethyl acetate (10 mL). The combined organic layers were dried over magnesium sulfate, filtered, and concentrated under reduced pressure. The crude material was purified using a Teledyne Gold 80 g column pre-equilibrated with 1% methanol/dichloromethane. The product eluted in 30% methanol/dichloromethane over several fractions, which were combined and concentrated. The material was redissolved in 1:1 water/acetonitrile (5 mL), frozen, and placed on lyophilizer to obtain 161 mg (30% yield) of a light purple solid.

LC/MS (Zorbax, SB-C18, StableBond, 4.6×150 mm, 5μm at 15-60% acetonitrile/water with 0.05% TFA): single peak, [M+H]+=597.3

### Conversion of MP-3854 to MMC

A 4 mg/mL solution of MP-3854 was made in ethanol. 40 mg of cysteine HCl was dissolved in 10 mL DI water (pH 2.1), and the pH was adjusted to 6.95 with a sodium hydroxide solution. 0.25 mL of the ethanol solution of MP-3827 was added to 2.5 mL of the cysteine solution, and the reaction was monitored by HPLC. The solution was heated to 37° C. in a heating block. Periodically, samples were removed for HPLC analysis, i.e., 25 μL sample was added into 0.45 mL methanol and then 1 mL DI water, and analyzed by HPLC at 360 nm. After 2 h, complete conversion was observed, generating 92% MMC (Area %).

### Remote Loading of MP-3854 into Liposome

21.1 mg of MP-3854 was weighed into a glass scintillation vial. To this was added 5 mL 0.3 M sucrose solution (filtered through 0.22 micron filter membrane) and the pH adjusted to 4.9. 5 mL of the solution was heated to 60° C. in a water bath. 5 mL of DSPC:Cholesterol:DSPE-PEG(2000) 55:40:5 with encapsulated ammonium citrate was heated to 60° C. in a second scintillation vial. At 60° C., the solution of MP-3854 was added all at once to the liposome and stirred at 60° C. for 20 min. The crude reaction was allowed to cool and then purified by passage through a Sepharose column (ca. 250 mL) using normal saline to elute the liposome. The liposome fraction was collected and concentrated using a 50 kDa centrifuge filter at 4000 rpm and at 4° C.

Assay: 25 μL of liposome was added to 475 μL of methanol followed by 1 mL of DI water. A clear solution was analyzed by HPLC at 360 nm and compared against a 4 mg/mL standard. The liposome contained 1.7 mg/mL MP-3854 with a particle size of 103.5 nm (volume mean).

### Example 6. 2-(pyridin-2-yldisulfanyl)ethyl (1aS,8S,8aR,8bS)-6-amino-8- ((carbamoyloxy)methyl)-8a-methoxy-5-methyl-4,7-dioxo-1a,4,7,8,8a,8b- hexahydroazirino[2′,3′:3,4]pyrrolo[1,2-α]indole-1(2H)-carboxylate) (MP-3830)

### Synthesis of MP-3830

MP-3830 was synthesized as described below:

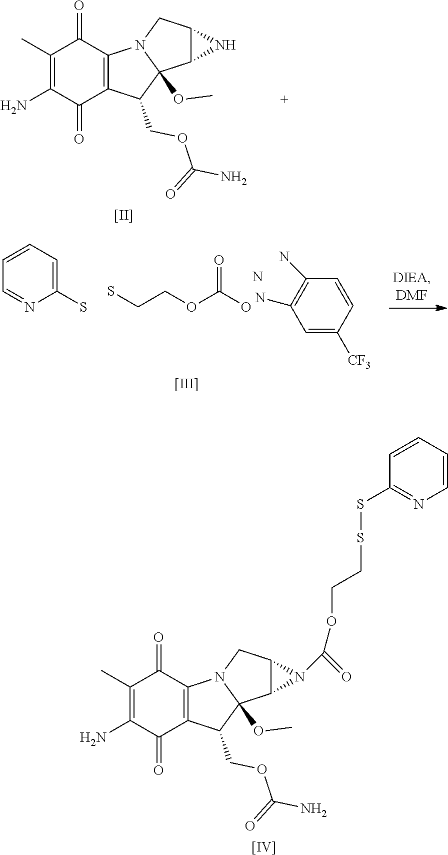

To a solution of MMC (184 mg, 0.550 mmol) and MP-3819 (229 mg, 0.550 mmol) in DMF (5 ml) was added DIEA (0.096 ml, 0.550 mmol). The mixture was stirred at RT. At 30 min., the reaction was analyzed by LC/MS (Zorbax, SB-C18, StableBond, 4.6×150 mm, 5 u at 15-60% acetonitrile/water with 0.05% TFA), which indicated that approximately 55% of the desired product (m/z=548.2) was formed. The reaction was diluted with ethyl acetate (7 ml) and washed with saturated sodium chloride (25 ml). The aqueous layer was extracted with ethyl acetate, and the combined organic layers were dried over anhydrous magnesium sulfate. Filtration and evaporation of the solvent under reduced pressure revealed a dark purple oil. The residue was dissolved in dichloromethane (4 mL), and the material was purified using a Teledyne Gold 40 g column (pre-equilibrated with 3 CV 1% methanol/dichloromethane). Elution was accomplished with 1% methanol/dichloromethane for 5 min followed by 5% methanol/dichloromethane. The product-containing fractions were combined and concentrated to a dark purple film which was then triturated with hexane. The solvent was decanted, and the product was dried under high vacuum to yield a dark purple solid (160 mg, 50% yield).

LC/MS (Zorbax, SB-C18, StableBond, 4.6×150 mm, 5 μm at 15-60% acetonitrile/water with 0.05% TFA): single peak at 8.33 min., [M+H]+=548.2

### Preparation of Liposome (Glutathione)

0.768 g glutathione was dissolved in 10 mL of Milli-Q water (bubbled with nitrogen) in a 10-mL volumetric flask. The pH of the 250 mM glutathione was adjusted using 10 N sodium hydroxide and 1 N sodium hydroxide to a pH of 7.03.

DSPC (149 mg), cholesterol (53 mg), and DSPE-PEG(2000) (48 mg) were weighed into a 100 mL round bottomed flask to which was added 10 mL of chloroform:methanol (3:1). This lipid solution was concentrated in vacuo at 40° C. 10 mL of glutathione solution was warmed to 70° C., and added to the flask containing the lipid film at 70° C. then mixed with a rotary mixer at 70° C. for 15 to 30 min.

The crude vesicles were then extruded through double stacked 200 nm membranes (Lipex 100 mL extruder) at 70° C. to produce a uniform particle size of approximately 200 nm (nitrogen used, 5 times at 200 psi). The liposome mixture was then extruded through double stacked 100 nm membranes to produce a uniform particle size of approximately 100 nm. This was done under pressure (nitrogen used, 5 times at 400 psi).

Liposomal glutathione was purified by Sepharose column chromatography using 0.9% saline (bubbled w/nitrogen) to elute the liposome fraction from the column. The liposome fraction from the Sepharose column was concentrated to approximately 10 mL using a centrifugal filtration unit (Amicon® Ultra, Centrifugal filters, Ultracel®-100K) at 10° C. and 4000 rpm.

The 10 mL liposome fraction was placed into a 10 mL clear serum bottle, sealed, labeled, and stored under a blanket of nitrogen in the walk-in cooler. Volume mean=100.9 nm, pH=6.95

### Remote Loading of MP-3830 in Liposome (Glutathione)

33 mg of MP-3830 was weighed into a glass scintillation vial. To this was added 0.8 mL ethanol and then 7.4 mL 0.3 M sucrose solution (filtered through 0.22 micron filter membrane). 8.2 mL of this solution was heated to 60° C. in a water bath. 8.2 mL of DSPC:Cholesterol:DSPE-PEG(2000) 55:40:5 with encapsulated glutathione was heated to 60° C. in a second scintillation vial. At 60° C., the solution of MP-3830 was added all at once to the liposome and stirred at 60° C. for 20 min. The crude reaction was allowed to cool and then purified by passage through a Sepharose column (ca. 250 mL) using normal saline to elute the liposome. The liposome fraction was collected and concentrated using a 50 kDa centrifuge filter at 4000 rpm and at 4° C.

Assay: 25 μL of liposome was added into 475 μL methanol followed by 1 mL DI water. A clear purple solution was analyzed by HPLC at 360 nm. The liposome contained 2.8 mg/mL MP-3861 with a particle size of 100.9 nm (volume mean).

### Reaction of MP-3830 with Glutathione (Preparation of MP-3861)

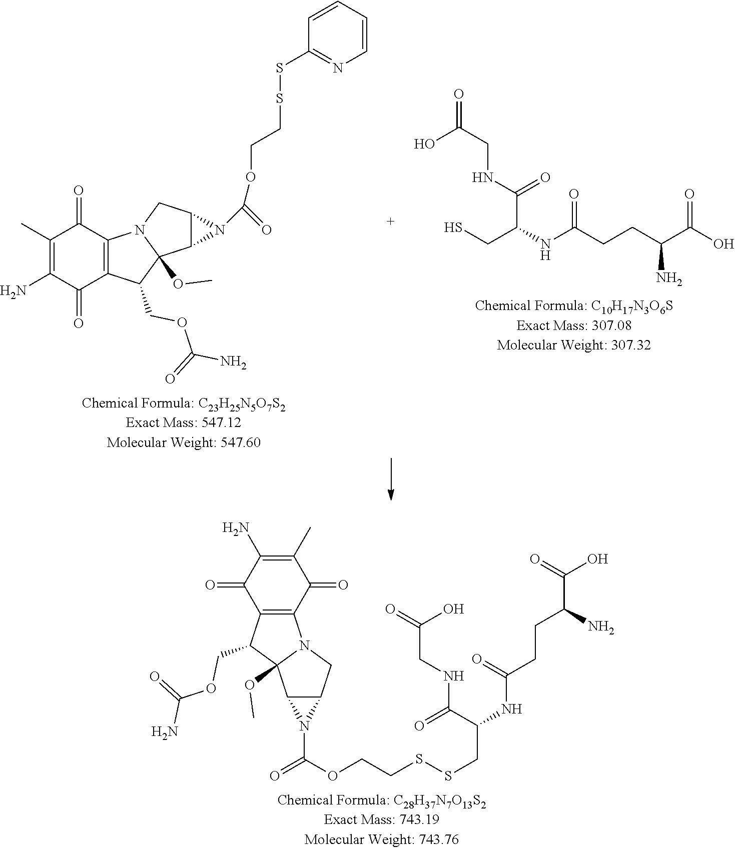 [SMILES: CO[C@@]12[C@H](COC(N)=O)C3=C(C(=O)C(C)=C(N)C3=O)N1C[C@H]1[C@@H]2N1C(=O)OCCSSc1ccccn1 ; N[C@@H](CCC(=O)N[C@H](CS)C(=O)NCC(=O)O)C(=O)O ; CO[C@@]12[C@H](COC(N)=O)C3=C(C(=O)C(C)=C(N)C3=O)N1C[C@H]1[C@@H]2N1C(=O)OCCSSC[C@@H](NC(=O)CC[C@H](N)C(=O)O)C(=O)NCC(=O)O]

44 mg of MP-3830 was weighed into a glass scintillation vial. To this purple semi-solid was added 1.5 mL ethanol and then 5 mL DI water. To the solution was added 25 mg glutathione in 5 mL DI water.

Assay: 25 uL of the mixture was diluted into 1 mL DI water. The solution was analyzed by HPLC at 360 nm. The crude reaction mixture was purified on a Gilson Prep HPLC with 10-70% ACN in water (0.10 M TEAA) over 20 min on waters column at 80 mL/min at 360 nM. Fractions were checked by LC/MS, and pure fractions were combined. Purified material was then lyophilized giving 33 mg of purple fluffy solid. LC/MS m/z=744.3 ([M+H]+)

### Example 7. Cryo-TEM of Liposomal MMC Prodrugs

Samples of each of the above-described liposomes (MP-3827, MP-3854 and MP-3861) were analyzed by Cryo-TEM with representative images shown below.

### Example 8. Liposomal MMC Prodrug Release Studies

### Preparation of Liposomal MP-3861 (Sample ID 289)

204 mg of MP-3830 was weighed into a glass scintillation vial. To this was added 5 mL ethanol, sonicated to a purple solution, and then 15 mL of 0.3M sucrose was added. The solution was heated to 60° C. in a water bath. 50 mL of DSPC:Cholesterol:DSPE-PEG(2000) 55:40:5 with encapsulated glutathione was heated to 60° C. in a 100 mL serum bottle. At 60° C., the solution of MP-3830 was added all at once to the liposome and stirred at 60° C. for 20 min. The crude reaction mixture was allowed to cool and then purified by passage through a Sepharose column (ca. 250 mL) using normal saline to elute the liposome. The liposome fraction was collected and concentrated using a 50 kDa centrifuge filter at 4000 rpm and at 4° C.

Assay: 25 μL of liposome was added to 475 μL methanol followed by 1 mL DI water. A clear purple solution was analyzed by HPLC at 360 nm against a standard at 4 mg/mL.

### Preparation of Liposomal MP-3854

159.1 mg of MP-3854 was weighed into a 100 mL serum bottle and 40 mL of 0.3M sucrose (insoluble) was added. To this, 1N HCl was added carefully to make a purple homogeneous solution. The pH was adjusted to 5.8 and the solution was heated to 60° C. in a water bath.

40 mL of DSPC:Cholesterol:DSPE-PEG(2000) 55:40:5 with encapsulated ammonium citrate was heated to 60° C. in a second 100 mL serum bottle. At 60° C., the solution of MP-3854 was added all at once to the liposome and stirred at 60° C. for 20 min.

The liposomal solution was divided into 3 portions:

A) 30 mL for diafiltration against histidine

B) 30 mL for diafiltration against acetate buffer

C) 20 mL for Sepharose column

### Sample ID 306A:

30 mL of liposome was diafiltered against 10 volumes of 20 mM histidine in saline at pH 6. The diafiltered material was concentrated to about 20 mL.

### Sample ID 306A-1

10 mL of diafiltered material was filtered through 0.2 micron syringe filters into pre-sterilized vials.

### Sample ID 306A-2

10 mL of diafiltered material was pH adjusted to 7 using 1N NaOH solution and then filtered through 0.2 micron syringe filters into pre-sterilized vials.

### Sample ID 306B

30 mL of liposome was diafiltered against 10 volumes of 20 mM acetate buffer in saline at pH 5. The diafiltered material was concentrated to about 20 mL, then filtered through 0.2 micron syringe filters into pre-sterilized vials.

### Sample ID 306C

20 mL of the liposomal mixture was purified by passage through a Sepharose column (ca. 250 mL) using normal saline to elute the liposome. The liposome fraction was collected and concentrated using a 50 kDa centrifuge filter at 4000 rpm and at 4° C., then filtered through 0.2 micron syringe filters into pre-sterilized vials.

Assay: 25 μL of liposome was added into 475 μL methanol followed by 1 mL DI water. A clear solution was analyzed by HPLC at 360 nm against a standard at 4 mg/mL.

Table 1 provides a summary of the physical properties of the liposomes.

TABLE 1
Summary of Liposomal Properties

| SAM- | | | | MMC | Total | |
|---|---|---|---|---|---|---|
| PLE | | | Particle | prodrug | lipids | Lipid:Pay- |
| ID# | Payload | Buffer | Size | (mg/ml) | (mg/ml) | load |
| 289 | MP- 3861 | Saline | 109.5 | 2.37 | 21.65 | 9.1 |
| 306A-1 | MP- 3854 | 20 mM | 99.7 | 3.05 | 17.47 | 5.7 |
| | | Histidine | | | | |
| | | pH 6 | | | | |
| 306A-2 | MP- 3854 | 20 mM | 98.8 | 2.97 | 17.49 | 5.9 |
| | | Histidine | | | | |
| | | pH 7 | | | | |
| 306B | MP- 3854 | 20 mM | 98 | 2.46 | 14.40 | 5.9 |
| | | Acetate | | | | |
| | | pH 5 | | | | |
| 306C | MP- 3854 | Saline | 102.4 | 2.56 | 13.56 | 5.3 |

### Release of MMC from Liposomal Mitomycin C Prodrugs in FBS

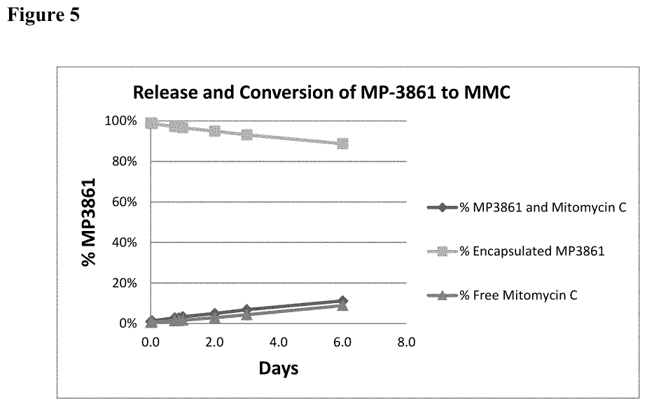

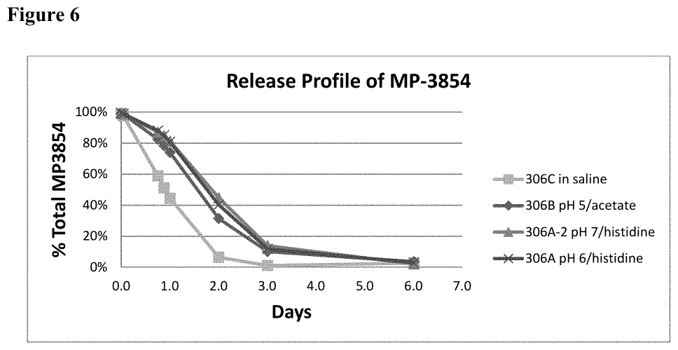

Samples were prepared in fetal bovine serum (FBS) (50 μL of liposomal sample diluted with 450 μL of FBS) and placed in a shaker at 37° C.; two such solutions were prepared for each sample. Aliquots were drawn at intervals over six days. The amount of liposomal MMC prodrug was determined by adding methanol to an aliquot of the sample (to break the liposome), while the free MMC was determined by using a spin-filter (Amicon centrifugal filter) to separate the free MMC from the proteins and liposomes. The prodrug amounts measured were assumed to be encapsulated, not free, at each time point. FIG. 5 shows the release of glutathione-entrapped MP-3861 from the liposome and its conversion to MMC in FBS. FIG. 6 shows the release profile of MP-3854 in FBS as a function of the buffer and pH.

### Example 9. In Vitro Cell Kill Assay

MCC, MMC prodrugs, and liposomal MMC prodrugs were evaluated in an in vitro cell kill assay. MMC, MMC prodrug, and liposomal MMC prodrug samples were diluted in DPBS as a starting concentration, then serially diluted 1:3 in DPBS (i.e., 70 μL:140 μL). HT29 colorectal adenocarcinoma cells were plated at 100 μL/well in 10% FBS/McCoy's 5a+Pen/Strep media at 5×10⁴ cells/mL, based on approximated 24-h doubling time. The plates were incubated at 37° C. overnight to allow the cells to adhere. Next, 10 μL/well treatment dose of MMC, MMC prodrug, or liposomal MMC prodrug was added to the wells, and the plates were incubated at 37° C. Cell killing ability was determined following either a 2-h, 24-h, or 72-h drug incubation. After a 2-h incubation, the media was removed from the plate and 100 μL/well drug-free media was added before returning to the incubator. After a 24-h incubation, the media was removed from the plate and 100 μL/well drug-free media was added before returning to the incubator. After a 72-h incubation, the media was removed from all of the plates and 100 μL/well drug-free media with 10% Alamar was added to each and returned to the incubator.

Cell viability was measured using WST-1 cell proliferation colorimetric assay or AlamarBlue. 50 mL of 10% WST/McCoy's 5a media was prepared from 5 mL of WST+45 mL of media; 50 mL of 10% Alamar Blue/McCoy's 5a media was prepared from 5 mL of Alamar Blue+45 mL of media. 10% WST/McCoy's 5a media or 10% Alamar Blue/McCoy's 5a was added, and the plates were returned to the incubator to develop, followed by subsequent reading of the fluorescence or absorbance.

The results of the in vitro cell kill assay are provided in Tables 2-5 below.

TABLE 2
Liposomal MP-3827 In Vitro Kill Data

IC50 (μM)

| Payload | 2 h | 24 h | 48 h | 72 h |
|---|---|---|---|---|
| Liposomal MP-3827 | 48.508 | 5.336 | 2.818 | 3.767 |
| MP-3827 | 5.934 | 2.641 | 1.686 | 1.294 |
| MMC | 1.833 | 0.175 | 0.109 | 0.199 |

TABLE 3
Liposomal MP-3854 - In Vitro Kill Data

| | IC50 (μM) | | |
|---|---|---|---|
| Payload | 2 h | 24 h | 72 h |
| Liposomal MP-3854 | >200 | 44.45 | 21.03 |
| MP-3854 | 41.45 | 4.17 | 3.09 |
| MMC | 1.833 | 0.175 | 0.199 |

TABLE 4
Liposomal MP-3832 - In Vitro Kill Data

| | IC50 (μM) | | |
|---|---|---|---|
| Payload | 2 h | 24 h | 72 h |
| Liposomal MP-3832 | >100 | 5.51 | 4.34 |
| MP-3832 | 4.70 | 1.44 | 1.71 |

TABLE 5
Glutathione-Entrapping Liposomal MP-3861- In Vitro Kill Data

| | IC50 (μM) | | |
|---|---|---|---|
| Payload | 2 h | 24 h | 72 h |
| Liposomal MP-3861 | >100 | 29.0 | 27.4 |
| MP-3861 | 12.5 | 1.06 | 0.902 |

### Example 10. Pharmacokinetics for Liposomal MP-3854 and MP-3861 in Mice

The plasma pharmacokinetics of liposomal MMC prodrugs and free MMC were studied in mice. The liposomal MMC prodrugs are described in Table 6 below.

TABLE 6
Liposome Sample Information for Pharmacokinetic Study

| | | | | particle | | Total | |
|---|---|---|---|---|---|---|---|
| | | | | size | | mg/mL | |
| Lipids | Payload | Salt | Buffer | (nm, vol.) | mg/mL | Lipids | Lipid:Drug |
| DSPC:Chol:DSPE-PEG(2000) | MP-3854 | Citrate | 20 mM | 104.5 | 2.91 | 25.6 | 8.8 |
| 55:40:5 | | | histidine/saline | | | | |
| DSPC:Chol:DSPE-PEG(2000) | MP-3861 | Glutathione | Saline | 106.2 | 4.97 | 26.5 | 5.3 |
| 55:40:5 | | adduct | | | | | |

Animals were randomized into five (5) groups, each with 18 mice. Each animal was given a single intravenous dose of liposomal MMC Prodrug or free MMC as shown in Table 7 below.

TABLE 7
Treatment Groups and Dosing Assignment

| | | | | | Mice |
|---|---|---|---|---|---|
| | | | Dose | | per |
| | | Dose | MMC- | | Time |
| | | MMC-eq | Prodrug | Time | Point |
| Group | Treatment | (mg/kg) | (mg/kg) | Points | (n) |
| 1 | MMC | 5 | — | 5 min, 4 h, 24 h, | 3 |
| | | | | 48 h, 96 h | |
| 2 | MP-3854 | 5 | 9 | 5 min, 4 h, 24 h, | 3 |
| | | | | 48 h, 96 h | |
| 3 | MP-3854 | 25 | 45 | 5 min, 4 h, 24 h, | 3 |
| | | | | 48 h, 96 h | |
| 4 | MP-3861 | 5 | 11 | 5 min, 4 h, 24 h, | 3 |
| | | | | 48 h, 96 h | |
| 5 | MP-3861 | 16 | 36 | 5 min, 4 h, 24 h, | 3 |
| | | | | 48 h, 96 h | |

Three animals were sacrificed at the appropriate time points post injection. Blood samples were taken for pharmacokinetic analysis at each time point (Table 8). Approximately 0.4 mls of blood was collected, via cardiac puncture under isoflurane anesthesia, into a lithium heparin tube and placed on ice. The samples were spun to obtain the plasma, which were stored in microfuge tubes at −80° C.

TABLE 8
Pharmacokinetic Parameters of Liposomal MMC Prodrug and Free MMC

| | Dose | Cmax | Half-Life | AUCₐₗₗ | Cl_obs | Vss_obs | Cmax_D |
|---|---|---|---|---|---|---|---|
| Compound | (mg/kg) | (ug/ml) | (hr) | (hr*ug/ml) | (ml/hr/kg) | (ml/kg) | (kg*ug/ml/mg) |
| MP-3854 | 9 | 199.8 | 6.3 | 2188.8 | 4.1 | 29.9 | 22.2 |
| MP-3854 | 45 | 740.5 | 8.5 | 11610.5 | 3.8 | 38.1 | 16.5 |
| MP-3861 | 11 | 169.8 | 12.2 | 3205.7 | 3.4 | 48.1 | 15.4 |
| MP-3861 | 36 | 734.2 | 11.1 | 15064.6 | 2.4 | 35.1 | 20.4 |
| Mitomycin C * | 5 | 1.2 | LLD | 20.4 | ND | ND | 0.24 |
| (Low dose of MP-3854) | | | | | | | |
| Mitomycin C * | 25 | 4.9 | LLD | 73.3 | ND | ND | 0.20 |
| (High dose of MP-3854) | | | | | | | |
| Mitomycin C * | 5 | 0.6 | Data not analyzed; plasma values too low | | | | |
| (Low Dose of MP-3861 | | | | | | | |
| Mitomycin C * | 16 | 2.6 | 16.3 | 65.4 | 213.9 | 4599.5 | 0.16 |
| (High Dose of MP-3861) | | | | | | | |

LLD = lower limit of detection
ND = not determined

### Example 11. In Vivo Anti-Tumor Activity of Liposomal MMC Prodrugs in Mice Bearing HT29 Xenograft Tumors

The anti-tumor activity of liposomal MMC prodrugs were evaluated in female Hsd:Athymic Nude-Foxn1^{nu/nu} mice (approximately 25 g) implanted subcutaneous with HT29 colorectal adenocarcinoma cells or (1×10⁶). Once tumors reached a median size of 200 mm³, animals were randomized into five (5) groups, normalized by tumor volume. Animals without tumors were not included in this study. Saline, MMC, and liposomal MMC prodrug were dosed intravenously as a single dose (not to exceed 30 ml/kg).

According to NCI euthanasia guidelines, mice were removed from the study if they loss 20% of their initial bodyweight or became moribund or if their tumor volume exceeded 2500 mm³ or if the tumor ulcerated. Tumor volume was measured with the TumorImager system at least twice per week and calculated with the TumorManager software, and mice were weighed twice per week.

Tumor volume was expressed as mean and median and plotted as a function of time. Animals removed from the study due to excess tumor size beyond 2500 mm³ had values carried forward as 2500 mm³ in the median plot only. Groups with less than 50% animals remaining were not carried forward for further tumor analysis. The following parameters were determined from tumor size and survival analysis: tumor growth inhibition (TGI), tumor growth delay (TGD), and median survival. The ratio of treated versus control tumor volume (% T/C) was calculated as:

$$\% T / C = \frac{mean tumor volume treatment group}{mean tumor volume control group} \times 100$$

Statistical significance of observed differences between growth curves were evaluated by One-Way ANOVA followed by posthoc test if significant.

A summary of the in vivo efficacy studies of liposomal MMC prodrug compared to MMC in mice bearing HT29 colorectal adenocarcinoma xenografts is shown in Table 9 below.

TABLE 9
Summary from in vivo Efficacy Studies in Mice

| | | MMC | | Dose | Day 7 | Day 28 | |
|---|---|---|---|---|---|---|---|
| Sample ID# | Lipids | mg/ml | Dose mpk | schedule | Survival | Survival | % T/C |
| MMC | | 0.5 | 2 | q7d x3 | 5/5 | 4/5 | 72 |
| | | | 5 | single | 5/5 | 4/5 | 77 |
| | | | 10 | single | 0/5 | 0/5 | — |
| | | 0.5 | 3 | q7d x3 | 5/5 | 5/5 | 13 |
| | | | 5 | q7d x3 | 5/5 | 3/5 | 5 |
| MP-3827 | DSPC:Chol:DSPE-PEG(2000) | 1.76 | 15 | single | 5/5 | 5/5 | 94 |
| | 55:40:5 | | 30 | single | 5/5 | 5/5 | >100 |
| | | 4.27 | 45 | single | 5/5 | 5/5 | 48 |
| | | | 90 | single | 0/5 | 0/5 | — |
| | | | 30 | q7d x3 | 5/5 | 5/5 | 18 |
| | | 5.1 | 60 | single | 1/5 | 1/5 | — |
| MP-3830/glutathione | DSPC:Chol:DSPE-PEG(2000) | 3.29 | 140 | single | 0/5 | 0/5 | — |
| | 55:40:5 | 2.8 | 60 | single | 5/5 | 5/5 | 29 |
| | | 5.2 | 90 | single | 2/5 | 0/5 | — |
| MP-3832 | DSPC:Chol:DSPE-PEG(2000) | 3.43 | 60 | single | 4/5 | 4/5 | 82 |
| | 55:40:5 | | 120 | single | 4/5 | 0/5 | — |
| MP-3854 | DSPC:Chol:DSPE-PEG(2000) | 2.06 | 60 | single | 5/5 | 4/5 | 30 |
| | 55:40:5 | 3.23 | 90 | single | 3/5 | 3/5 | 16 |
| | | 3.23 | 30 | q7d x3 | 5/5 | 5/5 | 53 |

These results indicate that these MMC prodrug liposomal compositions will improve the therapeutic index of MMC by improving its half-life and tolerability (i.e., reduce toxicity).

The MMC prodrug liposomal formulation with glutathione trapping exhibited sustained, slower release of the MMC prodrug and conversion to the active MMC.

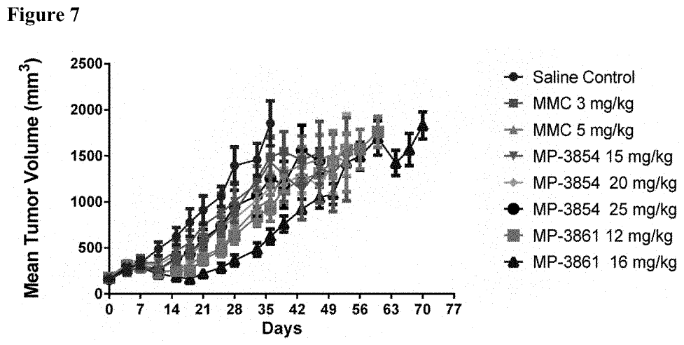

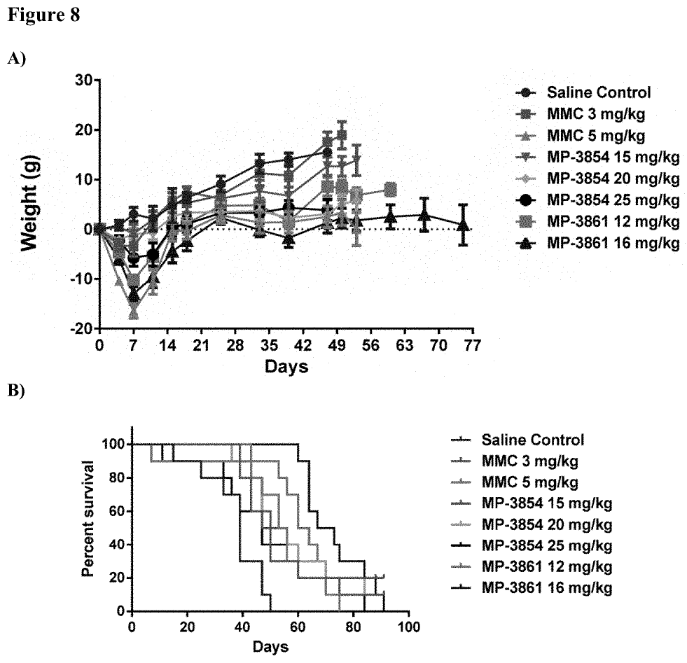

In another study, mice bearing HT29 colorectal adenocarcinoma xenografts were given a single intravenous dose of liposomal MMC Prodrugs (MP-3854 or MP-3861), free MMC, or saline and the results are summarized FIGS. 7 and 8 and Table 10 below.

TABLE 10
Treatment Groups, Dosing Assignments, and Efficacy Summary of Liposomal
MMC in Mice Bearing HT29 Colorectal Adenocarcinoma Xenografts

| | | | Dose | | | | | |
|---|---|---|---|---|---|---|---|---|
| | | | (mg/kg) | Dose | | | TGD | Median |
| | | Drug | MMC | (mg/kg) | Mice | | (1000 mm3 | Survival |
| Formulation | Dose | (mg/ml) | Prodrug | MMC-eq | (n) | % T/C | Days) | (Days) |
| Saline | single | | | | 10 | 100 | | 39 |
| MMC | single | 0.5 | | 3 | 10 | 83 | 5.2 | 48 |
| MMC | single | 0.5 | | 5 | 10 | 48 | 9.5 | 54 |
| MP-3854 | single | 2.87 | 27 | 15 | 10 | 74 | 4.8 | 53 |
| MP-3854 | single | 2.87 | 36 | 20 | 10 | 69 | 9.2 | 52 |
| MP-3854 | single | 2.87 | 45 | 25 | 10 | 68 | 9.6 | 47 |
| MP-3861 | single | 5.04 | 27 | 12 | 10 | 47 | 15.5 | 62 |
| MP-3861 | single | 5.04 | 36 | 16 | 10 | 31 | 23.2 | 70 |

The results demonstrate an increased efficacy of liposomal MMC prodrug compared to MCC (5 mg/kg).

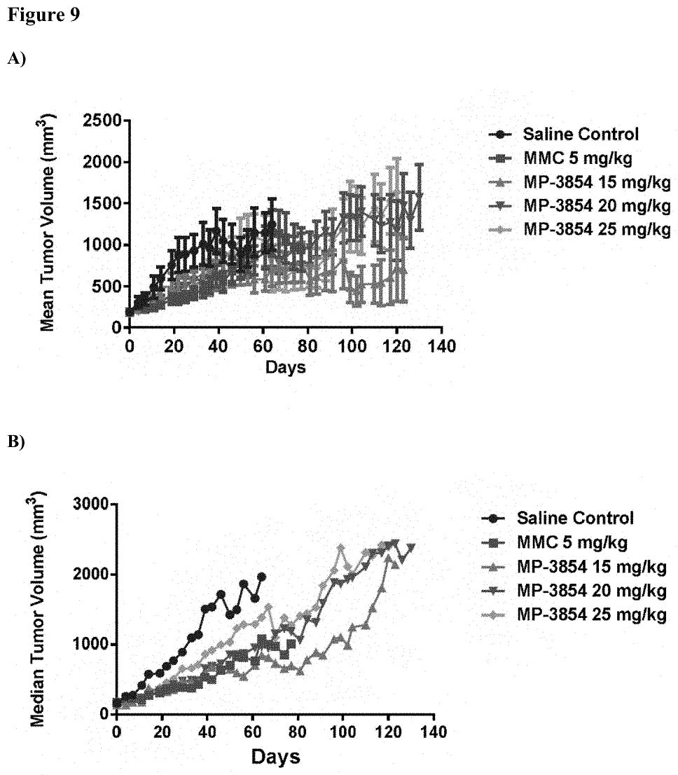

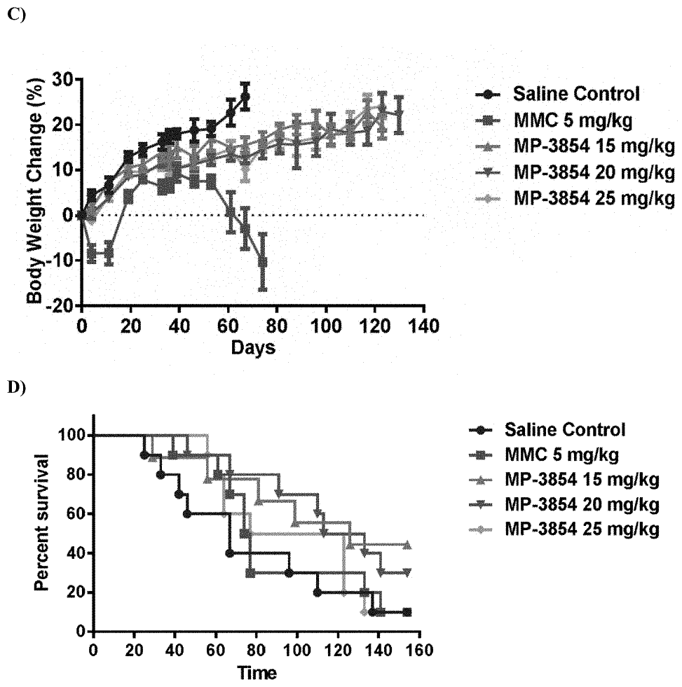

In yet another study, mice bearing A549 NSCLC xenografts were given a single intravenous dose of liposomal MMC Prodrug (MP-3854), free MMC, or saline and the results are summarized in FIG. 9 and Table 11 below.

TABLE 11
Treatment Groups, Dosing Assignments, and Efficacy Summary
of Liposomal MMC in Mice Bearing A549 NSCLC Xenografts

| | | | Dose | | | | | |
|---|---|---|---|---|---|---|---|---|
| | | | (mg/kg) | Dose | | | TGD | Median |
| | | Drug | MMC | (mg/kg) | Mice | | (1000 mm3 | Survival |
| Formulation | Dose | (mg/ml) | Prodrug | MMC-eq | (n) | % T/C | Days) | (Days) |
| Saline | single | | | | 10 | 100 | | 67 |
| MMC | single | 0.5 | | 5 | 10 | 90 | 32 | 76 |
| MP-3854 | single | 2.87 | 27 | 15 | 10 | 59 | 63 | 126 |
| MP-3854 | single | 2.87 | 36 | 20 | 10 | 76 | 37 | 123 |
| MP-3854 | single | 2.87 | 45 | 25 | 10 | 104 | 15 | 100 |

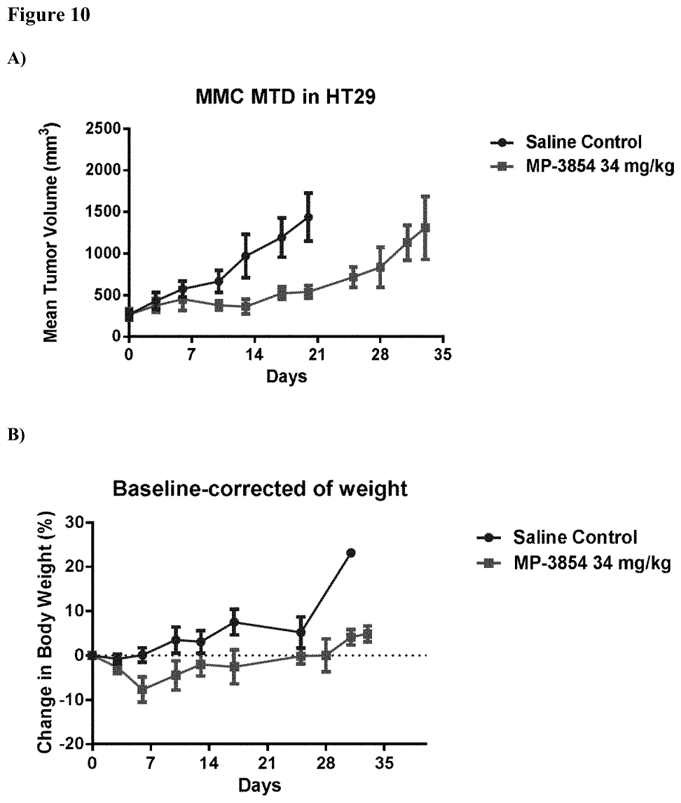

In another study, mice bearing HT29 colorectal adenocarcinoma xenografts were given a single intravenous dose of liposomal MMC Prodrug (W-3854) or saline and the results are summarized FIG. 10 and Table 12 below.

TABLE 12
Treatment Groups, Dosing Assignments, and Efficacy
Summary of Liposomal MMC in Mice Bearing HT29
Colorectal Adenocarcinoma Xenografts

| | | | Dose | | | | |
|---|---|---|---|---|---|---|---|
| | | | (mg/kg) | Dose | | | |
| Formu- | | Drug | MMC | (mg/kg) | Mice | % | 28 day |
| lation | Dose | (mg/ml) | Prodrug | MMC-eq | (n) | T/C | Survival |
| Saline | single | | | | 5 | 100 | 2/5 |
| MP-3854 | single | 2.87 | 60 | 34 | 5 | 37 | 4/5 |

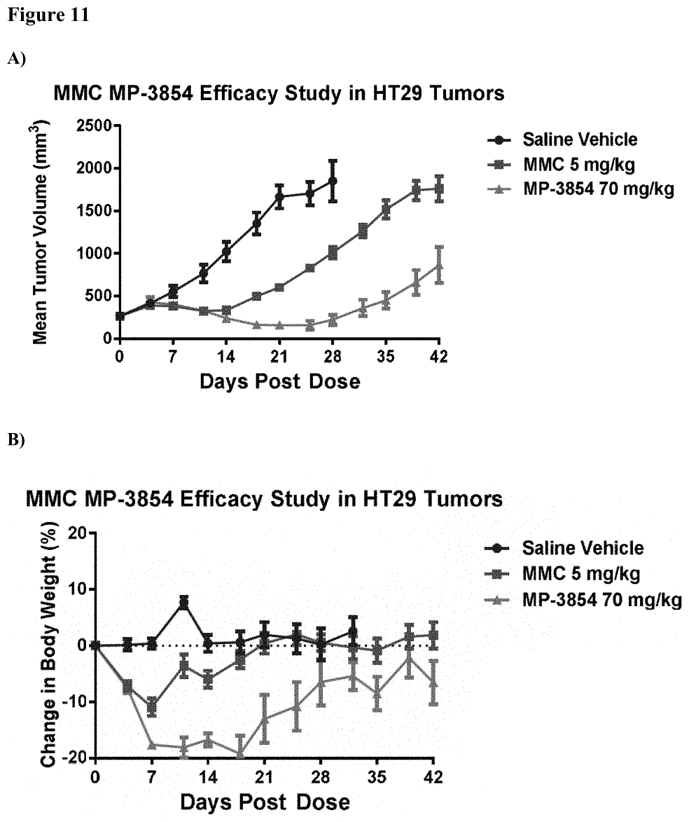

In a further study, mice bearing HT29 colorectal adenocarcinoma xenografts were given a single intravenous dose of liposomal MMC Prodrug (W-3854) or saline and the results are summarized FIG. 11 and Table 13 below.

TABLE 13
Treatment Groups, Dosing Assignments, and Efficacy
Summary of Liposomal MMC in Mice Bearing HT29
Colorectal Adenocarcinoma Xenografts

| | | | Dose | | | |
|---|---|---|---|---|---|---|
| | | | (mg/kg) | Dose | | |
| | | Drug | MMC | (mg/kg) | Mice | 28 day |
| Formulation | Dose | (mg/ml) | Prodrug | MMC-eq | (n) | Survival |
| Saline | single | | | | 5 | 5/5 |
| MP-3854 | single | 3.6 | 125 | 70 | 5 | 4/5 |

Although the foregoing has been described in some detail by way of illustration and example for purposes of clarity and understanding, one of skill in the art will appreciate that certain changes and modifications can be practiced within the scope of the appended claims. In addition, each reference provided herein is incorporated by reference in its entirety to the same extent as if each reference was individually incorporated by reference.

1. A compound having the formula: 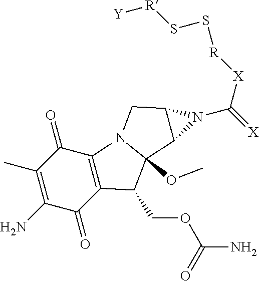
wherein R and R′ are independently selected from the group consisting of: an alkyl and alkylaryl;
X is selected from the group consisting of: S, Se, and O; and
Y is selected from the group consisting of: a piperadinyl, a piperazinyl, a pyridinyl, dimethylamino, diethylamino, dipropylamino, morpholino, HO—CH₂CH₂NH—, (HO—CH₂CH₂)₂N—, HO—CH₂CH₂—N(Me)—, C₆H₄CH₂NH—, and C₆H₄CH₂N(Me).

2. A compound having the formula selected from the group consisting of: 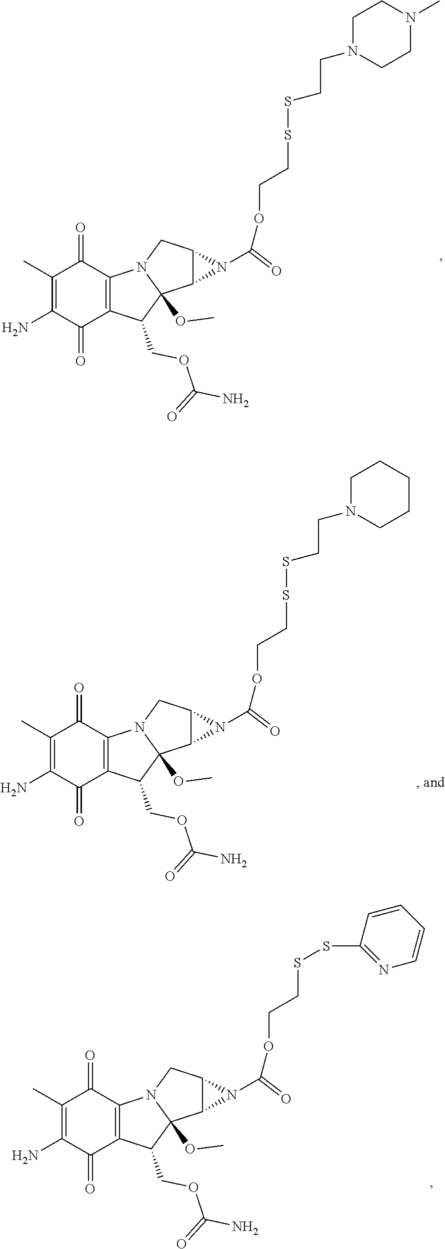
or a pharmaceutical salt thereof.

3. (canceled)

4. (canceled)

5. A liposomal composition comprising:
i) a liposome comprising:
a) a phosphatidylcholine lipid,
b) a sterol,
c) a PEG-lipid, and
d) a MMC prodrug; and
ii) a pharmaceutically acceptable excipient;
wherein the MMC prodrug is a compound having the formula: 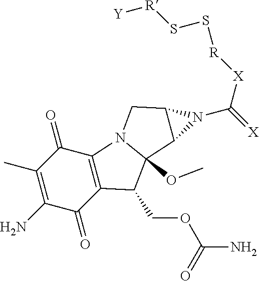
wherein R and R′ are independently selected from the group consisting of: an alkyl and alkylaryl;
X is selected from the group consisting of: S, Se, and O; and
Y is selected from the group consisting of: a piperadinyl, a piperazinyl, a pyridinyl, dimethylamino, diethylamino, dipropylamino, morpholino, HO—CH₂CH₂NH—, (HO—CH₂CH₂)₂N—, HO—CH₂CH₂—N(Me)—, C₆H₄CH₂NH—, and C₆H₄CH₂N(Me).

6. The liposomal composition of claim 5, wherein the MMC prodrug is selected from the group consisting of: 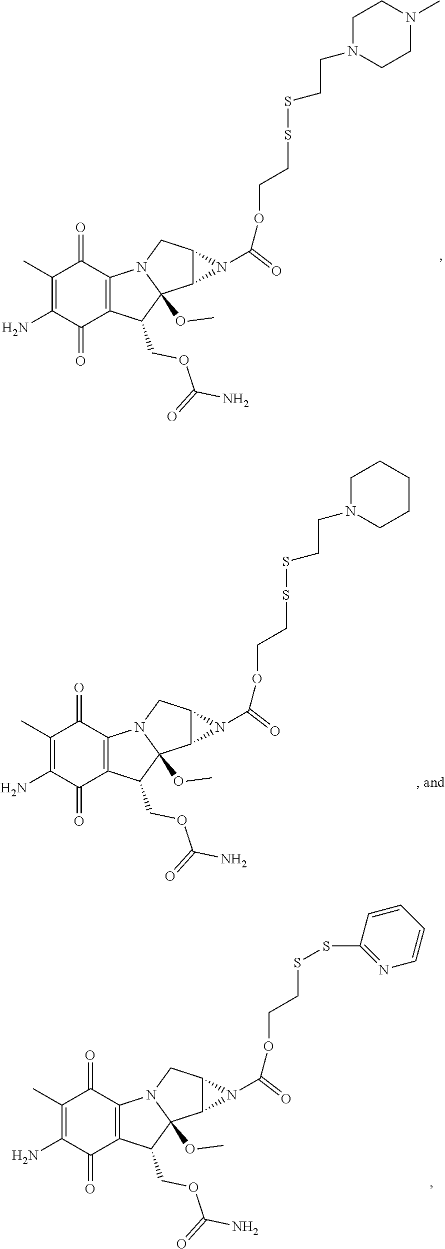
or a pharmaceutical salt thereof.

7. A method of preparing a liposomal MMC prodrug, the method comprising:
a) forming a first liposome having a lipid bilayer comprising a phosphatidylcholine lipid and a sterol, wherein the lipid bilayer encapsulates an interior compartment comprising an aqueous solution; and
b) loading the first liposome with a MMC prodrug, or a pharmaceutically acceptable salt, to form a loaded liposome;
wherein the MMC prodrug is a compound having the formula: 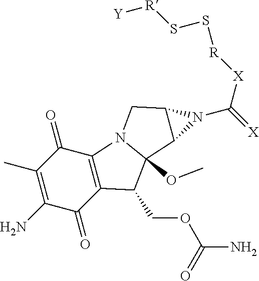
wherein R and R′ are independently selected from the group consisting of: an alkyl and alkylaryl;
X is selected from the group consisting of: S, Se, and O; and
Y is selected from the group consisting of: a piperadinyl, a piperazinyl, a pyridinyl, dimethylamino, diethylamino, dipropylamino, morpholino, HO—CH₂CH₂NH—, (HO—CH₂CH₂)₂N—, HO—CH₂CH₂—N(Me)—, C₆H₄CH₂NH—, and C₆H₄CH₂N(Me);
thereby forming the liposomal MMC prodrug.

8. (canceled)

9. The method of claim 7, wherein the interior compartment of the first liposome further comprises a thiol-containing compound.

10. The method of claim 9, wherein the MMC prodrug and thiol-containing compound undergo a disulfide exchange resulting in a MMC prodrug conjugate.
